(19) United States
(12) Patent Application Publication
(10) Pub. No.: US 2024/0159767 A1
(43) Pub. Date: May 16, 2024

(54) GENETICALLY ENCODED SYNTHETIC REACTION-DIFFUSION SYSTEM THAT CAN GENERATE PROGRAMMABLE OSCILLATIONS, PATTERNS, AND SPATIOTEMPORAL SIGNALING CIRCUITS IN MAMMALIAN CELLS

(71) Applicant: Wisconsin Alumni Research Foundation, Madison, WI (US)

(72) Inventors: Scott Coyle, Madison, WI (US); Rohith Rajasekaran, Madison, WI (US)

(21) Appl. No.: 18/508,825

(22) Filed: Nov. 14, 2023

Related U.S. Application Data
(60) Provisional application No. 63/425,294, filed on Nov. 14, 2022.

(51) Int. Cl. G01N 33/68

(52) U.S. Cl. CPC G01N 33/6803

(57) ABSTRACT

Compositions and methods for generating and utilizing spatial and temporal distributions of molecules are disclosed. The effect is based on the observation that certain proteins that serve a spatial and/or temporal function in their native environments can be expressed in different environments to achieve different spatial and/or temporal distributions. This observation showed that whole-cell oscillations, traveling waves, spirals, turbulent patterns, static patterns, and other patterns could be generated and observed. These spatial and temporal distributions were then coupled with functional moieties, which provided the functionality in the same spatial and temporal distributions. The model system for these observations is expression of MinD and MinE proteins in Eukaryotic cells.

### CROSS-REFERENCE TO RELATED APPLICATIONS

This application is related to, claims priority to, and incorporates by reference for all purposes U.S. Provisional Patent Application No. 63/425,294, filed Nov. 14, 2022.

### STATEMENT REGARDING FEDERALLY SPONSORED RESEARCH

N/A

### SEQUENCE LISTING

The contents of the electronic sequence listing (20960004_WARF-0002-U01.xml; Size: 196 kbytes; and Date of Creation: Nov. 8, 2023) accompanying this application are herein incorporated by reference in their entirety. An informal sequence listing was filed with U.S. Provisional Patent Application No. 63/425,294 as Appendix C, which is incorporated herein in its entirety by reference. It is noted that the sequence numbering was adjusted and the numbering in the electronic sequence listing prevails for this document, though reference in the earlier filings will correspond to the sequence numbering in those filings.

### BACKGROUND OF THE INVENTION

Cell biology is animated by the dynamic organization of protein activities in space and time. By specifying when and where specific proteins act, cells can build a diverse range of functions needed for growth, information processing, and motility from a common set of conserved molecular components. Disruption of this spatiotemporal organization at the single-cell level can lead to human diseases, and bacteria and viruses often pattern host-cell activities in new ways to hijack host-cell biology. Thus, the spatiotemporal organization of proteins is a key programming language of cell biology that demands deeper understanding and greater engineerable control.

Despite its fundamental importance, there are few tools that enable us to artificially organize the spatiotemporal dynamics of proteins inside living cells. Two common strategies are localization sequences, which direct a protein of interest to a static location in the cell; and optogenetics, which allows an experimenter to organize molecules within an illuminated region of the cell. Although both approaches have significant utility, they force us to choose between genetic encodability and dynamic control. As such, they fail to recapitulate the self-organization that is a hallmark of all living cells.

Despite the critical importance of spatiotemporal dynamics in cell biology, the current landscape for genetically encoding these phenomena is limited. That is, there are almost no synthetic protein circuits that can robustly generate dynamic molecular patterns artificially in cells.

### BRIEF SUMMARY OF THE INVENTION

In an aspect, the present disclosure provides a composition or kit comprising one or more polynucleotides. The one or more polynucleotides include the sequence of MinD and/or MinE.

In another aspect, the present disclosure provides a method of directing the spatial and temporal distribution of molecules within a cell. The method includes expressing a first MinDE pair comprising a first MinD and a first MinE in a Eukaryotic cell.

In another aspect, the present disclosure provides a composition of matter including a cell. The cell non-natively expresses a first MinDE pair comprising a first MinD and a first MinE. The first MinD and/or first MinE is coupled to a reporter or functional molecule. The reporter or functional molecule generates a reporter or functional signal that is detectable at a frequency associated with a reaction diffusion frequency of the MinD and the MinE. The reaction diffusion frequency is tailored by an overall and relative concentration of the MinD and the MinE.

In a further aspect, the present disclosure provides a method of exploiting spatial and temporal distribution behaviors generated by non-natively expressed protein pairs. The method includes: observing a spatial and temporal distribution of molecules within a cell including whole-cell oscillations, traveling waves, spirals, turbulent patterns, static patterns, or combinations thereof being generated by a pair of proteins with a cell, wherein the pair of proteins is not natively expressed in the cell; and expressing the pair of proteins within the cell, wherein a functional moiety is coupled to one of the pair of proteins, thereby coupling the functional moiety to the spatial and temporal distribution of molecules within the cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is an illustration of how oscillating MinDE signal can act as a “cellular radio station” that imparts the cell with a unique genetically-encoded frequency.

FIG. 2 is a schematic illustration showing the MinD ATPase and its activator MinE form a protein-based reaction-diffusion system (MinDE) in E. coli that generates pole-to-pole oscillations to specify the division site. Transplanting these components into human cells provides a synthetic biology platform for engineering more complex spatiotemporal signaling circuits.

FIG. 3A includes example images, kymographs, and quantification of traveling waves, as described in Example 1.

FIG. 3B includes example images, kymographs, and quantification of traveling oscillations, as described in Example 1.

FIG. 3C includes example images, kymographs, and quantification of persistent stationary patterns, as described in Example 1.

FIG. 4 is an image-level and pixel-level time series of mCherry-MinD fluorescence data can be remapped to the frequency domain by Fast Fourier Transform (FFT) to produce image-level and pixel-level power spectra, as described in Example 1. Individual cells appear in the slice of the image-level power spectrum that corresponds to their unique MinDE cellular frequency ƒMinDE. Examples of these representations are shown for a pair of recently divided cells.

FIG. 5 shows frequency-domain image processing applied to the time-series data of a single fluorescent signal (mCherry-MinD). An example of spectral isolation of an individual cell with a 28 mHz MinDE frequency from its neighbors.

FIG. 6 shows generation of a multi-channel image in which each color represents a different MinDE frequency is shown, as described in Example 1. Cells can be located and segmented by taking advantage of their MinDE frequency barcode.

FIG. 7 is a representative image from a 24-hour timelapse of migrating 3T3 cells false-colored by MinDE frequency, as described in Example 1. Frequency-barcodes persist throughout the time course and enable spectral resolution even when cells physically overlap.

FIG. 8 shows an example (left) of spectral separation of MinDE signals occurring in different sub-compartments of the same cell. The pixel-level power spectrum of mCherry-MinD signals collected over a 6-hour time series revealed distinct frequencies corresponding to separate nuclear and cytoplasmic MinDE oscillations. Using Finite Impulse Response (FIR) filters based on the power spectrum in the left panel, the nuclear and cytoplasmic signals were isolated, and the Hilbert Transform was used to estimate the instantaneous power of each signal throughout the 6-hour recording. This produces a multi-channel representation of the data (right) that separately labels and tracks the cytoplasmic and nuclear signals. Identical results were obtained using Continuous Wavelet Transform.

FIG. 9 is a schematic representation illustrating how thousands of MinDE circuit configurations sampling different expression levels or component ideas can be characterized in parallel using the frequency-domain image processing techniques described herein.

FIG. 10 is a series of data plots. Aggregating single-cell circuit behavior across 1000 s of U2OS cells reveals how MinDE circuit frequency (left panel) and MinDE circuit power (middle panel) can be programmed at a genetic level by controlling the expression levels of MinD and MinE. The minE:MinD expression ratio (right panel) is the major determinant of MinDE circuit frequency. This enables characterization and comparison of the frequency-scaling behavior of MinDE circuits harboring different mutant components. The frequency-scaling behavior for MinE mutants that affect MinE membrane affinity and diffusivity are shown.

FIG. 11 is a schematic of a plug-and-play platform for inducible recruitment of protein payloads to MinDE circuits based on rapamycin-dependent interaction between FRB-MinD and FKBP-payloads.

FIG. 12 includes data illustrating the inducible recruitment of a simple BFP payload using the system described in Example 1. Representative FRB-mCherry-MinD and BFP-FKBP images of cells pre and post rapamycin inductions are shown, demonstrating rapamycin-dependent colocalization of BFP with MinD.

FIG. 13 includes power spectra for the MinD and BFP signals from FIG. 12 pre- and post-rapamycin induction. MinD shows a consistent high-power signal in the presence or absence of rapamycin. The BFP signal has little power at the MinD frequency in the absence of rapamycin, but high-power (13× increase) when rapamycin is added.

FIG. 14 shows MinDE frequency scaling behavior for the MinD-BFP recruitment circuit in the presence or absence of rapamycin. There is little change in the steady-state frequency of the MinD signal upon recruitment.

FIG. 15 includes representative images, pixel-level intensity time course, and kymograph pre and post recruitment of the condensate-forming intrinsically-disordered region of FUSN to MinDE. The MinDE signal undergoes rapid frequency modulation as puncta-like structures assemble and disassemble along the wave trajectory.

FIG. 16 is similar to FIG. 15 but using a pre-formed protein condensate (FTH-FUSN) as a payload. MinDE oscillations immediately arrest as MinDE components colocalize into droplets.

FIG. 17 is a schematic summarizing how MinDE signaling circuit behaviors and dynamics can be genetically programmed and further modulated in real time by engineered protein-protein interactions.

FIG. 18 is a schematic representation showing MinDE circuits can be engineered to receive, barcode, and broadcast the dynamics of any fluorescent cell-state reporter. A specific circuit that broadcasts PKA kinase signaling activity was designed by fusing a PKA substrate to MinD and co-expressing FHA1-mCerulean, which binds only to the phosphorylated form of that PKA substrate. This PKA→MinDE→FHA1 circuit creates a new signal derived from MinDE (the carrier) in the mCerulean channel that is modulated by PKA signaling activity (the data line).

FIG. 19 is an example of pixel-level read-out of PKA signaling activity in a 293T cell using the circuit in FIG. 18. Upon treatment with the PKA agonist isoprenaline, the FHA1 signal (PKA data line) begins to co-oscillate with MinDE (carrier signal). Normalization of the power of the FHA1 signal to the MinDE carrier recovers the real-time PKA signaling dynamics for that pixel within the cell.

FIG. 20 shows represenative images (left) of a population of cells bearing the circuit in FIG. 18, showing the power of the MinD (carrier line) or FHA1 (PKA data line) signals pre or post treatment with isoprenaline, color-coded by frequency. Upon treatment with isoprenaline, the PKA data line lights up at the corresponding frequency of the MinD carrier for each cell. 98 single-cell PKA signaling trajectories extracted using the frequency-barcoded data broadcast by the cells are clustered by response behavior (right).

FIG. 21 is a schematic illustration of how MinDE circuits can also be engineered to act as control signals that drive cellular activities. A specific circuit that inducibly couples MinDE spatiotemporal dynamics to actin polymerization upon rapamycin induction was designed using the bacterial signaling effector ActA as an FKBP-payload.

FIG. 22 is a demonstration that the MinDE→ActA control signal circuit can organize the polymerization of actin into structures with position and shape dictated by the MinDE signal, using a stationary MinDE pattern as an example control signal. Representative images for MinD (control signal) and the LifeAct actin reporter (output signal), pre (−rap) and post (+rap) coupling are shown, along with a kymograph passing through multiple MinDE spots.

FIG. 23 shows an example cell bearing a MinDE→ActA circuit driven by a dynamic MinD wave. When MinDE control signals are dynamic, they can be used to probe signaling timescales within the cell. Associated kymograph is also shown.

FIG. 24 shows a single-pixel intensity time course (left) of the MinD and LifeAct signals, which indicates that the LifeAct (output signal) consistently lags behind the MinD (control signal). A representative pixel-level cross-correlation profile (right) between MinD and ActA signals and associated image-level rendering of the extracted time-lag across the field of view in FIG. 23. This shows a consistent 8 s lag between LifeAct and MinD everywhere within the cell, defining the timescale for signal transmission from ActA to actin polymerization.

FIG. 25 is a binary plot of functionality between MinD and MinE of various species, as described in Example 2.

FIG. 26 is a frequency plot of functionality between MinD and MinE of various species, as described in Example 2.

FIG. 27 is a power plot of functionality between MinD and MinE of various species, as described in Example 2.

### DETAILED DESCRIPTION OF THE INVENTION

Provided herein are methods and compositions for controlling the spatial and temporal distribution of molecules within a cell. In particular directing the spatial and temporal distribution by a genetically encoded circuit.

In some embodiments the genetically encoded circuit comprises components of the MinD/ParA family of ATPases. The Min System is a mechanism composed of three proteins MinC, MinD, and MinE used by bacteria as a means of properly localizing the septum prior to cell division. Other bacterial positioning proteins include MipZ, PomZ SpoIIE, DivIVA, RacA, TipN, PopZ, RuBisCo. In particular embodiments, MinD and MinE are used to enable spatiotemporal patterns.

Some embodiments comprise MinD and MinE polynucleotides.

The Min system, including MinD and MinE, is not natively found in Eukaryotic cells. Given the very specific way in which they function in Prokaryotic environments, expressing these proteins in Eukaryotic cells is a highly unpredictable venture. The inventors surprisingly discovered that MinD and MinE could be expressed in a Eukaryotic cell to provide a spatial and temporal distribution of molecules (e.g., the MinD and MinE, themselves) within the cell. Moreover, the inventors surprisingly discovered that the particular spatial and temporal distribution could be tailored by way of adjusting the overall concentration of MinD and MinE, as well as the relative concentrations of the two. This discovery led to other important discoveries, as articulated below, which relate to exploiting this behavior of MinD and MinE to provide useful functions within a cell.

Without wishing to be bound by any particular theory, it is believed that the absolute and relative concentrations of MinD and MinE can be adjusted to provide a desired spatial and temporal distribution. In certain cases, the absolute and relative concentration of MinD and MinE is selected to cause the spatial and temporal distribution of molecules to have a characteristic frequency.

In some cases, the MinD and/or MinE can be coupled to a payload, which has the effect of providing the payload at the same or a related spatial and temporal distribution as the MinD and/or MinE to which the payload is coupled.

In some cases, the payload is a reporter molecule, such as a fluorescent tag, which allows visualization of the MinD and/or MinE spatial and temporal distribution. Without wishing to be bound by any particular theory, the visualization effect serves as an excellent proof of principle, as some of the functional molecules may not be as readily observable as the reporter molecules. Absent compelling biochemical reasoning to the contrary, it is believed that a functional payload should behave in a similar fashion to the reporter payloads described herein, and therefore, the successful showing of an effect using reporter molecules could be expected to have reasonably predictable results with most functional molecules. In other words, the likelihood of success of achieving the proof-of-principle and other validating experiments described herein was low in the minds of the inventors (and would have been low in the minds of an ordinarily skilled artisan), but once the effect was observed with reporter molecules, the likelihood of success of replacing the reporter molecules with functional molecules and achieving a similar effect was much higher, because the underlying principle was much better understood following the initial experiments.

In some cases, the payload is a functional molecule, which allows the function of that functional molecule to be distributed in the same spatial and temporal distribution as the MinD and/or MinE to which the functional molecule is coupled. For example, if the MinD and MinE are tailored to a given cellular environment to provide a standing sine-wave distribution of MinD and MinE, then if a functional molecule is coupled to either MinD or MinE, then functional molecule will also be provided in the cell in the standing sine-wave distribution. Similarly, if the MinD and MinE are tailored to give a specific frequency modulation at a given location in a cell, then the functional molecule will also be provided in the cell at that given specific frequency modulation at that given location.

The frequency-specific nature of the MinD and MinE behavior can be controlled and utilized to allow selective observation of cells that have been tailored to provide different unique frequencies. Using the tools described herein, a population of cells can be provided which have different spatial and temporal distributions of MinD and MinE, thereby allowing the cells to be probed to determine their identity externally using optical techniques, such as lock-in amplification. For example, a mixture of three different cell types could be modified with the systems described herein, such that the MinD and MinE total and relative concentrations are adapted to provide three distinct frequencies. Those distinct frequencies can be tracked to provide real-time cellular identification.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

The term “construct” or “polynucleotide construct” is a polynucleotide which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the construct is an expression construct, a vector or a viral vector. A vector is any particle used as a vehicle to artificially carry a foreign nucleic sequence, typically DNA into another cell, where it can be replicated and/or expressed. A vector containing foreign DNA is termed recombinant DNA. The four major types of vectors are plasmids, viral vectors, cosmids, and artificial chromosomes. Expression constructs comprise a heterologous promoter and the nucleic acid sequence encoding protein of interest (e.g., MinDE) which is capable of expression in the cell in which it is introduced. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”) and may be used interchangeably. Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the mutant α-gal protein. Another type of vector is a viral vector, wherein additional DNA segments may be ligated into the viral genome. Viral vectors are incorporated into viral particles that are then used to transport the viral polynucleotide encoding the protein of interest into the target cells. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses (AAV)), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV), among others and includes constructs that are able to express the protein of interest in cells. A preferred vector is a lentiviral vector or adeno-associated vector. Suitable methods of making viral particles are known in the art to be able to transform cells in order to express the protein of interest in mammalian cells described herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be a plant, animal, bacterial, fungal, or synthetic promoter. Suitable promoters are known and described in the art.

Suitable promoters for expression in plants include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

Within the vector may be an expression cassette. An expression cassette is a distinct component of vector DNA consisting of a gene and regulatory sequence to be expressed by a transfected cell. A vector may further comprise a kozak consensus sequence, one or more linkers, one or more tags and one or more promoters. Linker sequences are known in the art and may comprise glycine and/or serine. Exemplary vectors, vector components and sequences are included herein and/or in the accompanying sequence listing. For example baseline circuits, and components are included in SEQ ID NO: 1 through SEQ ID NO: 29 and SEQ ID NO: 54 through SEQ ID NO: 79. Exemplary activity circuits, components are included in SEQ ID NO: 30 through SEQ ID NO: 36. Exemplary Payload circuits and components are included in SEQ ID NO: 37 through SEQ ID NO: 53.

Exemplary sequences for MinD and MinE, including as MinDE pairs, are described herein.

In some cases, the MinD is Escherichia coli MinD having an amino acid sequence of MARIIVVTSGKGGVGKTTSSAAIATGLAQKGKKTVVIDFDIGLRNLDLIMGCERRVVYD FVNVIQGDATLNQALIKDKRTENLYILPASQTRDKDALTREGVAKVLDDLKAMDFEFIV CDSPAGIETGALMALYFADEAIITTNPEVSSVRDSDRILGILASKSRRAENGEEPIKEHLLL TRYNPGRVSRGDMLSMEDVLEILRIKLVGVIPEDQSVLRASNQGEPVILDINADAGKAY ADTVERLLGEERPFRFIEEEKKGFLKRLFGG (SEQ ID NO: 54). In some cases MinE is Escherichia coli MinE having an amino acid sequence of

(SEQ ID NO: 67)
MALLDFFLSRKKNTANIAKERLQIIVAERRRSDAEPHYLPQLRKDILEVI
CKYVQIDPEMVTVQLEQKDGDISILELNVTLPEAEELK.

In some cases, the MinD is Fukatsuia symbiotica MinD having an amino acid sequence of MARIIVVTSGKGGVGKTTSSAAIATGLAQQGKKTVVIDFDIGLRNLDLIMGCERRVVYD FVNVIQEDATLNQALIKDKRTENLYILPASQTRDKDALTKEGVEKVLTGLDEMNFEYVV CDSPAGIETGALMALYFADEAIITTNPEVSSVRDSDRILGILSSKSRRAKRGEEPIKEHLLL TRYNPSRVSRGDMLSMEDVLDILRIPLLGVIPEDQSVLSASNQGEPVILNGGSNAGKAYA DTVARLLGEKRNFRFIEEEKKGFLKRLFGG (SEQ ID NO: 55). In some cases, the MinE is Fukatsuia symbiotica MinE having an amino acid sequence of

(SEQ ID NO: 68)
MALLDFFLSGKKPTANIAKERLQIIVAERRRGEQEPHYLPDLKRDVLAVI
CKYVQINPDMLQVQFEQKGDDISVLELNVTLPDMEETSK.

In some cases, MinD is Gullanella endobia MinD having an amino acid sequence of MVRIIVVTSGKGGVGKTTSSAAIATGLARKGKKTVVIDFDIGLRNLDLIMGCERRVVYD FINVINCDATLNQALIKDKRTENLYVLPASQTRDKDALTREGVEKVLNDLDTMEFDFIIC DSPAGIETGALMALYFADEAIITTNPEISSVRDSDRILGILSAKSRRAENGLEAIKEHLLLT RYNPRRVKCGDMLSMRDVIEILRIPLLGLIPEDQSVLRASNQGEPVILDKESDAGQAYID MVDRLLGEEHPFRFIEEEKKGFIKRLFRG (SEQ ID NO: 56). In some cases, MinE is Gullanella endobia MinE having an amino acid sequence of MALLNFFISRKKSTANIAKERLQIIVAEQRRGNNEPHYLPQLKRDLIEVINKYVQIDSKML SMQLEKDGNISILELNIALLETEESIE (SEQ ID NO: 69).

In some cases, MinD is Moranella endobia MinD having an amino acid sequence of MARIIVVTSGKGGVGKTTSSASIATGLARKGKKTVVIDFDIGLRNLDLIMGCERRVVYDF VNVVQGAARLTQALIKDKRTDNLYILPASQTKDQDALTCAGVEKLLNDLNKMEFDFIV CDSPAGIETGALMALYFADEAIITTNPEVSSVRDSDRILGILSSKSRRAEIGQEPIKEHLLL NRYNPGRVSRGDMLSIDDVIEILRIPLVGVIPEDQSVLQASNQGEPVILNEDSDAGQAYS DMVDRLLGQECPFRFIAEPQKKSLLKRLFGV (SEQ ID NO: 57). In some cases, MinE is Moranella endobia MinE having an amino acid sequence of

(SEQ ID NO: 70)
MVLIDFFLARKKNTANIAKERLHSIVAERRGSNEPYYLPQLKSDLLEVIS
KYAKIDITMISIQLDQKDENLSILELNVKLTDTH.

In some cases, MinD is Clostridium tertium MinD having an amino acid sequence of MGVSIVITSGKGGVGKTTTTANIGTALAALNKRVVVVDGDTGLRNLDVLMGLENRIVY TITDVIENRCRLKQALIKDKRYQNLCLLPTAQTKDKDDIRPQDMLKLINELKEDFDYVLI DCPAGIEQGFENSVVGADRAVVVVNPEITSVRDADRVIGKLDAKGLDDHAVIINRLNYE MTQRGDMLDVSDIIETLSIELLGVVPDDKNITVSTNKGEPIVLDDKSISGQAFKNIARRIT GEEVPLLDLKTGGEGFFASIKRLFKR (SEQ ID NO: 58). In some cases, MinE is Clostridium tertium MinE having an amino acid sequence of

(SEQ ID NO: 71)
MEFLRKLSSRPTPKEVAKDRLKLILIHDRGDLPHETLEKIRMEILEVLSK
YIEIDSEDVEIAVSKTENVEGNNPALVANIPIKNIK.

In some cases, MinD is Dickeya dadantii MinD having an amino acid sequence of MARIIVVTSGKGGVGKTTSSAAIATGLAQKGKKTVVIDFDIGLRNLDLIMGCERRVVYD FVNVIQNDATLNQALIKDKRTENLYILPASQTRDKEALTREGVDKVLKDLADMAFDFIIC DSPAGIETGALMALYFADEAIITTHPEVSSVRDSDRILGILSSKSRRAEQGQEPIKEHLLLT RYNPGRVSRGDMLSMEDVLEILRIPLVGVIPEDQSVLRASNQGEPVILDKDADAGKAYE DTVDRLLGEERPYRFIEEEKKSFLKRLFGG (SEQ ID NO: 59). In some cases, MinE is Dickeye dadantii MinE having an amino acid sequence of

(SEQ ID NO: 72)
MALLDFFLSRKKTTANIAKERLQIIVAERRRGDSEPHYLPQLKRDILEVI
CKYVQIDPEMVTVQLEQKGDDISVLELNVTLPEADEATPPTDK.

In some cases, MinD is Helicobacter felis MinD having an amino acid sequence of MVITITSGKGGVGKSTTTANLAIGLALQNKKVVAVDFDIGLRNLDMILGLENRIVYDVID VMEGNCKLPQALINDKKNKNLYFLPASQSKDKNILDKAKVQALIAQLNAQFDFVLIDSP AGIESGFEHAVLFADRAIIVVTPEVSSVRDSDRVIGIIDAKSCKGQEMVKHILINRIKPDLV EKQEMLSNEDVLKILALPLIGLVPEDDKIVSATNTGEPVIYTQSPSALAFQRITRRVLGEE VEFAEFRTKRGLVGTIKGWFA (SEQ ID NO: 60). In some cases, MinE is Helicobacter felis

MinE having an amino acid sequence of

(SEQ ID NO: 73)
MKWFKGGSSARARDRLTLVLAYERSMRIPYMEEMKKEILAVVQKYIATTK
IDVRTSSNQEMDTLEVEIILERNSKGNPES.

In some cases, MinD is Helicobacter pylori MinD having an amino acid sequence of MAIVVTITSGKGGVGKSTTTANLAIGLAESGKKVVAVDFDIGLRNLDMILGLENRIVYD VVDVMEKNCNLSQALITDKKTKNLSFLAASQSKDKNILDKEKVAILINALRADFDYILID SPAGIESGFEHAILHADMALVVVTPEVSSLRDSDRVIGIIDAKSNRAKKGMEVHKHLIINR LKPELVANGEMISIEEVLKILCLPLIGIIPEDSHIISATNKGEPVIRADCESAKAYQRITRRIL GEEVEYVEFKAKRGFFGALKGIFS (SEQ ID NO: 61). In some cases, MinE is Helicobacter pylori MinE having an amino acid sequence of

(SEQ ID NO: 74)
MSLFDFFKSKGSAATATDRLKLILAKERTLNLPYMEEMRKEIIAVIQKYT
KSSDIHFKTIDGNQSVETIEVEIILPK.

In some cases, MinD is Klebsiella quasipneumoniae MinD having an amino acid sequence of MARIIVVTSGKGGVGKTTSSAAIATGLAQKGKKTVVIDFDIGLRNLDLIMGCERRVVYD FVNVIQGDATLNQALIKDKRTENLYILPASQTRDKDALTREGVDKVLEELKKMEFDFIV CDSPAGIETGALMALYFADEAIITTNPEVSSVRDSDRILGILASKSRRAENGEEPIKEHLLL TRYNPGRVNKGDMLSMEDVLEILRINLVGVIPEDQSVLRASNQGEPVILDAASDAGKAY ADTVERLLGEERPFRFIEEEKKGFLKRLF (SEQ ID NO: 62). In some cases, MinE is Klebsiella quasipneumoniae MinE having an amino acid sequence of

(SEQ ID NO: 75)
MALLDFFLSRKKNTANIAKERLQIIVAERRRGDAEPHYLPQLRKDILEVI
CKYVQIDPEMVSVQLEQRDGDISILELNVTLPETEESKP.

In some cases, MinD is Pantoea sp. Mhis MinD having an amino acid sequence of MSRIIVVTSGKGGVGKTTSSAAIATGLAQNNKRTAVIDFDIGLRNLDLVMGCERRVVYD FINVIQGDATLNQALIRDKHTEQLYILPASQTRNKDALTRKGVEKVIQELEENDFDFIICD SPAGIEAGALMALYFADEAIIITNPEVSSVHDSDRILGIISSKSYRAENGKTPIKEYLLLTRY NPNRVTRGDMLSMEDVLGILRIPLLGVIPEDQSVLRSSNQGEPVILDTNSDAGKAYFDTV ERLLGKELPFRFIEEEKKGFIKRLFGG (SEQ ID NO: 63). In some cases, MinE is Pantoea sp. Mhis MinE having an amino acid sequence of

(SEQ ID NO: 76)
MALLKFFLSHKKNTANIAKERLQIIVAEHRKARNEPHYLPQLKRDILKVI
CKYVKINPEMVTVQLEHKQDNISILELNIALLEVKDLSNSNMI.

In some cases, MinD is Photobacterium damselae MinD having an amino acid sequence of MARIIVVTSGKGGVGKTTSSAAIASGLALRGKKTAVIDFDIGLRNLDLIMGCERRVVYDF VNVINGEANLNQALIKDKRTDNLFVLPASQTRDKDALSKEGVERVLKDLGEMGFDFVIC DSPAGIETGALMALYFADEAIVTTNPEVSSVRDSDRILGILDSKSRRAEQGQEPVKQHLL LTRYNPTRVNQGDMLSVQDVEEILHIPLLGVIPESQAVLNASNKGEPVIFDKDADASIAY QDTVARLLGEECPFRFLEEEKKGFLKRLFGG (SEQ ID NO: 64). In some cases, MinE is Photobacterium damselae MinE having an amino acid sequence of

(SEQ ID NO: 77)
MALLEFFRPKKTTTASVAKERLQIIVAERRSAGQGAPSYLPQLKQDILEV
IRKYVAIDPEQVVVTLDQKEEDLAVLELNVTLPEEDK.

In some cases, MinD is Pseudomonas poae MinD having an amino acid sequence of MAKILVVTSGKGGVGKTTTSAAIGTGLALRGHKTVIVDFDVGLRNLDLIMGCERRVVY DFVNVVNGEANLQQALIKDKRLENLYVLAASQTRDKDALTKEGVGKVLAELKETFEYV VCDSPAGIETGAHLAMYFADEAIVVTNPEVSSVRDSDRMLGLLASKSQRAEKGEEPIKE HLLLTRYNPERVNNGEMLGVEDVKDILAVTLLGVIPESQAVLKASNQGVPVILDDQSDA GQAYSDAVDRLLGKTVDHRFLDVKKKGFFERIFGGN (SEQ ID NO: 65). In some cases, MinE is Pseudomonas poae MinE having an amino acid sequence of

(SEQ ID NO: 78)
MKFLDFFRANKKPSTASVAKERLQIIVAHERGQRSTPDYLPALQKELVEV
IRKYVNIGNDDVHVALENDGSCSILELNITLPDR.

In some cases, MinD is Thorsellia anophelis MinD having an amino acid sequence of MTKVLVVTSGKGGVGKTTSSAAISTGLARKGKKTVVIDFDIGLRNLDLIMGCERRVVYD FVNVIQGDATLNQALIKDKRTENLYILPASQTRDKDALTRDGVEKVINELKEMEFDFIIC DSPAGIESGALMALYFADEAIITTNPEVSSVRDSDRILGILSSKSKRAEQGDNPIKEHLLIT RYNPTRVSHGDMLSMEDVLEILRVPLVGLIPEDQSVLRASNQGEPVILDENSDAGKAYD DMVARLLGEERQFRFLSEEKKSFLKRLFGG (SEQ ID NO: 66). In some cases, MinE is Thorsellia anophelis MinE having an amino acid sequence of

(SEQ ID NO: 79)
MSLINFFLSKKKNTANIAKERLQIIVAERRKADSAPAYLEEMKRDLLAVI
CKYVQIDPDMLHVGYEQKDDDISVLELNITLPENEEK.

In general, the sequences described herein can be varied slightly while maintaining their function. The BLAST algorithm is one exemplary method for determining sequence homology. In some cases, the systems and methods that utilize the sequences disclosed herein can include sequences having at least 95% homology, at least 98% homology, or at least 99% homology. Other methods of determining sequence homology focus on structural homology (e.g., adopting the same folded structure) with measured or modeled structures.

Ultimately, one significant test for homology in this case is the retention of a desired performance characteristic, as described herein.

It should be appreciated that while sharing identity is a strong indicator of shared function, there may be instances and likely will be instances where proteins having lower identity are capable of performing the same function. If species A and species B have low identity/low homology, this does not necessarily mean they are not capable of comparable function. Generally, sequences with 40-50% identity are likely to perform similar functions at a qualitative level. Indeed, many D or E sequences with 70% identity have been discovered to generate behaviors described herein, but often with some different quantitative behavior. At the same time, very high identity may not exhibit the same behaviors or may not function in all cases. In some cases, the issue may relate to expression in the particular environment (e.g., eukaryotic cell versus original bacterial host) or an evolutionarily selected feature of the protein that has impacted activity. Many of the observations described herein are empirically based and the process for making further such observations is described herein.

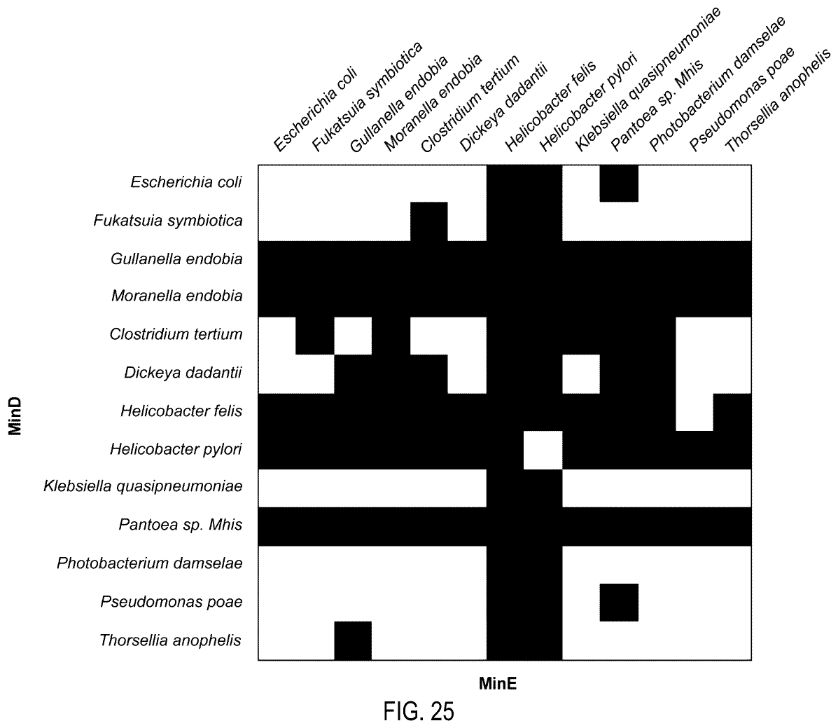

The disclosed MinDE pairs, disclosed MinD species, and disclosed MinE species are not an exhaustive list of all MinDE pairs, MinD species, or MinE species. It is believed that other species of bacteria may express MinDE pairs, MinD species, and/or MinE species. The experimental results provided herein suggest that most MinDE pairs include at least one MinD species or MinE species that is capable of forming a functioning MinDE pair with a respective MinE species or MinD species from the same or a different species. A skilled artisan will recognize that assessment of a new MinDE pair including a new MinD species and a new MinE species can involve cross-referencing the new MinD species against the new MinE species and all other known MinE species (e.g., those disclosed below in Example 2 at the time of drafting) and cross-referencing the new MinE species against all other known MinD species (e.g., those disclosed below in Example 2 at the time of drafting). In effect, a skilled artisan would understand the need to add a new row and a new column to FIGS. 25-27 and to conduct the straightforward experiment required to acquire the data to complete each of the newly-formed cells.

Without wishing to be bound by any particular theory, a particular combination of a MinDE pair can be a target for new discoveries, owing to the frequency spectrum produced by the pair. The different species components from the identified pairs can be combined in the fashion described elsewhere herein to produce new frequency/amplitude behaviors and to potentially provide new orthogonal pairings.

In order to form a functioning MinDE pair, a selected MinD and its corresponding MinE must have some reactivity with one another in order to achieve the behaviors described herein. One initially unexpected discovery is that MinDE pairs from a single species are not always functional. For example, it was unexpectedly discovered (note: unexpected following the initial discovery that underlies this disclosure—this is effectively a second layer of unexpected results on top of the initial discovery) that expressing MinD and MinE from G. endobia, expressing MinD and MinE from M. endobia, or expressing MinD and MinE from H. felis in a Eukaryotic cell does not produce observable behaviors as described herein. On the other hand, it was discovered that expressing MinD and MinE from each of E. coli, F. symbiotica, C. tertium, D. dadantii, H. pylori, K. quasipneumoniae, P. sp. Mhis, P. damselae, P. poae, and T. anophelis does produce the observable behaviors. It was not apparent in advance of these experiments which of these species would provide functioning MinDE pairs and which would not. Exemplary methods of determining if a MinDE pair is functioning are provided below in Example 2.

General criteria involve using sequence alignment tools, such as BLAST, iterative BLAST, or mmSEQ to find candidate sequences that align with a query sequence (for example, one of the sequences disclosed herein). One starting approach involves identifying MinD species and MinE species with over 50% identity and other features that are expected to benefit the behaviors described herein. These other features include, but are not limited to: i) the presence of a membrane-targeting-like sequence on the MinD and/or MinE sequences (suggesting interaction with membranes); and ii) the MinD and/or MinE sequences occur adjacent to one another on the chromosome (operon structure). By choosing sequences with differing identity percentages, sequences that are likely to function as oscillators are screened and there is the potential to uncover MinD and/or MinE species that exhibit different quantitative features and/or orthogonality with other MinDE pairs.

In some cases, diversity amongst the candidates is particularly desired (e.g., a lower identity/homology between candidates) to avoid redundancy. For example, if there are a dozen candidate sequences that have 70% identity to a known MinD or MinE species but the dozen candidates all have 99% identity to each other, then one of the dozen candidates can be selected as a representative candidate. In some cases, the search for new candidates involves a search for diverse and non-overlapping sequences.

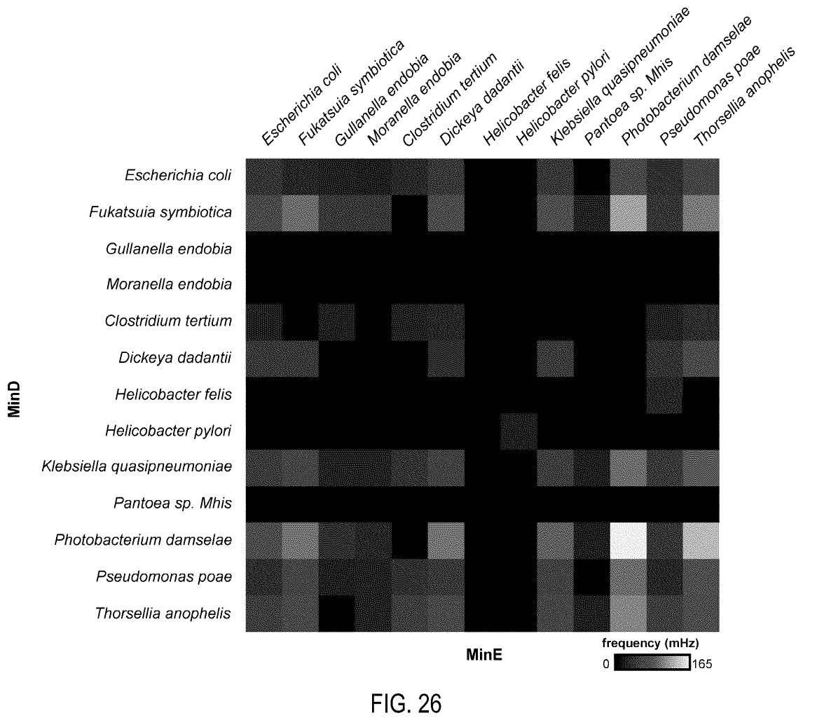

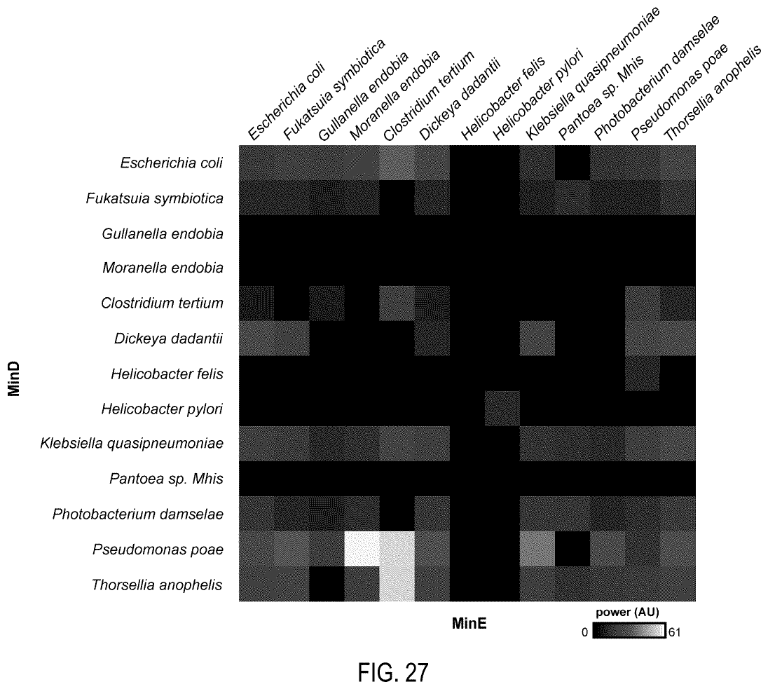

Cross-referencing between species was performed as outlined below in Example 2. The different MinDE pairs can undergo a binary screen for activity, as shown in FIG. 25, where white boxes represent active pairs and black boxes represent inactive pairs. The different MinDE pairs provided different characteristic frequencies, as shown in FIG. 26. The different MinDE pairs provided different power outputs, as shown in FIG. 27.

In addition to MinDE pairs from a single species exhibiting the behaviors described herein (though not universally, as discussed above), combinations of MinD from one species with MinE from a second species also exhibited the ability to express signals as described herein.

Without wishing to be bound by any particular theory, the following observations were made regarding combinations of MinD and MinE.

In some cases, certain MinD did not form a successful MinDE pair with any of the MinE that were sampled. Specifically, G. endobia MinD, M. endobia MinD, and Pantoea sp. Mhis MinD did not exhibit the behaviors described herein when expressed with any of the MinE specifically recited herein. It should be appreciated that these MinD species may be capable of pairing with MinE species that are not yet identified and may be capable of serving various inhibitory functions without interfering with circuitry (dependent on performance with other MinDE pairs). These MinD species also serve as evidence that the likelihood of success in this endeavor has a low baseline.

In some cases, certain MinE did not form a successful MinDE pair with any of the MinD that were sampled. Specifically, H. felix MinE did not exhibit the behaviors described herein when expressed with any of the MinD specifically recited herein. It should be appreciated that this MinE species may be capable of pairing with MinD species that are not yet identified and may be capable of serving various inhibitory functions without interfering with circuitry (dependent on performance with other MinDE pairs). These MinE species also serve as evidence that the likelihood of success in this endeavor has a low baseline.

In some cases, the first MinD or the first MinE has an amino acid sequence selected from the group consisting of SEQ ID NOs: 54-79. In some cases, the first MinD has an amino acid sequence selected from the group consisting of SEQ ID NOs: 54, 55, 58, 59, 60, 61, 62, 64, 65, and 66. In some cases, the first MinE has an amino acid sequence selected from the group consisting of SEQ ID NOs: 67, 68, 69, 70, 71, 72, 74, 75, 76, 77, 78, and 79.

The observation that reaction between MinD and MinE from varying species is not universal (e.g., some pairs do not function) has led to the discovery of some additional useful functions that could not have otherwise been observed. In some cases, a first MinDE pair including a first MinD and a first MinE can be orthogonal to a second MinDE pair including a second MinD and a second MinE. As used herein, an “orthogonal pair” of MinDE refers to a circumstance where all of the following conditions are true: the first MinD and first MinE are reactive and exhibit the behaviors described herein; the second MinD and the second MinE are reactive and exhibit the behaviors described herein; the first MinD and the second MinE are not reactive and do not exhibit the behaviors described herein; and the second MinD and the first MinE are not reactive and do not exhibit the behaviors described herein. The first MinDE pair is effectively an independent “circuit” from the second MinDE pair, because the reactants do not cross-interact with one another. As such, the first MinDE pair can be configured to provide a first operating behavior while the second MinDE pair is configured to provide a second operating behavior that is independent of the first.

The ability to couple function to the observable and reproducible patterns described herein opens the door to a wide variety of possibilities in cellular engineering. As one example, a first payload coupled to a first MinDE system can include molecular building blocks, thereby distributing the molecular building blocks in the pattern generated by the MinDE pair, and a second payload coupled to a second MinDE system can include assembling components for assembling the molecular building blocks into useful outputs. The result of this hypothetical system would be that the building blocks are provided in the pattern generated by the first MinDE pair, the assembling components are provided in the pattern generated by the second MinDE pair, and the useful output is provided in the regions where there is overlap, such that the assembling components and the molecular building blocks are co-localized. Without limit, the disclosed MinDE system may be applicable to: production of new cytoskeletal structures (actin or microtubules) with tunable geometries and modulable dynamics and mechanics; spatiotemporal organization of signaling components to change the INPUT/OUTPUT behavior of native or synthetic signaling pathways (e.g., modulating CAR T signaling, cancer cell signaling); or use the system to dynamically concentrate and deconcentrate components of a biosynthetic pathway to automate sequences of reactions.

Specifically relating to orthogonal pairs, without limit, the disclosed MinDE system allows multiple frequencies to be used within the same cell for massive multiplexable data encoding. In other words, a cell would have a “Channel A” and a “Channel B” at different frequencies that can be used for independent data encoding. Identifying more species and more orthogonal pairs will flesh out the toolkit for providing tailored behaviors.

It should be appreciated that these independent circuits can have different functional and/or reporter molecules coupled to a MinD or MinE involved in those circuits, which then conveys the independent nature of the MinDE pair control to the coupled functional and/or reported molecules. If a first MinDE pair provides a first characteristic signal and a second orthogonal MinDE pair provides a second characteristic signal that is independent of the first characteristic signal, then coupling a first functional moiety to the first MinDE pair as described elsewhere herein pair provides the first function provided by the first functional moiety in the pattern given by the first MinDE pair and provides the second function provided by the second functional moiety in the pattern given by the second MinDE pair. As discussed elsewhere herein, visualization molecules and methods can be used to first determine the pattern before functionally exploiting the pattern.

In some cases, a MinDE pair can be a more or less universally orthogonal, because the MinD from the pair is not reactive with any identified MinE other than the MinE from its shared species and vice versa. As one non-limiting example, the MinDE pair from H. pylori exhibits the behavior described herein when expressed in a Eukaryotic cell, but does not exhibit the behavior when expressing MinD or MinE with a respective MinE or MinD from a different species. As such, the MinDE pair from H. pylori can be considered a universal orthogonal pair relative to the sampled MinDE pairs.

In some cases, a MinD can pair with a MinE from a different species to form a pair that is more or less universally orthogonal.

In the methods and compositions described herein, where a MinDE pair, a MinD, and/or a MinE is discussed, it is impliedly referencing a first MinDE pair, a first MinD, and/or a first MinE. Without limitation, the present disclosure contemplates the use of a second MinDE pair comprising a second MinD and a second MinE, a third MinDE pair comprising a third MinDE and a third MinE, and an nth MinDE pair comprising an nth MinDE and an nth MinE. The pairs may be tailored to interact with one another or they may be tailored to be orthogonal to one another.

In some cases, the MinD and the MinE within a MinDE pair are from the same species. In some cases within a MinDE pair, the MinD is from one species and the MinE is from a second, different species.

Examples of suitable MinDE pairs that exhibit one or more of the behaviors described herein are shown below in Example 2. Referring to FIG. 25, each white square represents a functioning MinDE pair.

Focusing specifically on the MinD having an amino acid sequence including SEQ ID NO: 54, a functioning MinDE pair can be formed with a MinE having an amino acid sequence selected from the group consisting of SEQ ID NOs: 67, 68, 69, 70, 71, 72, 75, 77, 78, and 79. Focusing specifically on the MinE having an amino acid sequence including SEQ ID NO: 67, a functioning MinDE pair can be formed with a MinD having an amino acid sequence selected from the group consisting of SEQ ID NOs: 54, 55, 58, 59, 62, 64, 65, and 66.

Focusing specifically on the MinD having an amino acid sequence including SEQ ID NO: 55, a functioning MinDE pair can be formed with a MinE having an amino acid sequence selected from the group consisting of SEQ ID NOs: 67, 68, 69, 70, 72, 75, 76, 77, 78, and 79. Focusing specifically on the MinE having an amino acid sequence including SEQ ID NO: 68, a functioning MinDE pair can be formed with a MinD having an amino acid sequence selected from the group consisting of SEQ ID NOs: 54, 55, 59, 62, 64, 65, and 66.

Focusing specifically on the MinD having an amino acid sequence including SEQ ID NO: 56, no functioning MinDE pair can be formed using the MinE that were sampled. Focusing specifically on the MinE having an amino acid sequence including SEQ ID NO: 69, a functioning MinDE pair can be formed with a MinD having an amino acid sequence selected from the group consisting of SEQ ID NOs: 54, 55, 58, 62, 64, and 65.

Focusing specifically on the MinD having an amino acid sequence including SEQ ID NO: 57, no functioning MinDe pair can be formed using the MinE that were sampled. Focusing specifically on the MinE having an amino acid sequence including SEQ ID NO: 70, a functioning MinDE pair can be formed with a MinD having an amino acid sequence selected from the group consisting of SEQ ID NOs: 54, 55, 62, 64, 65, and 66.

Focusing specifically on the MinD having an amino acid sequence including SEQ ID NO: 58, a functioning MinDE pair can be formed with a MinE having an amino acid sequence selected from the group consisting of SEQ ID NOs: 67, 69, 71, 72, 78, and 79. Focusing specifically on the MinE having an amino acid sequence including SEQ ID NO: 71, a functioning MinDE pair can be formed with a MinD having an amino acid sequence selected from the group consisting of SEQ ID NOs: 54, 58, 62, 65, and 66.

Focusing specifically on the MinD having an amino acid sequence including SEQ ID NO: 59, a functioning MinDE pair can be formed with a MinE having an amino acid sequence selected from the group consisting of SEQ ID NOs: 67, 68, 72, 75, 78, and 79. Focusing specifically on the MinE having an amino acid sequence including SEQ ID NO: 67, a functioning MinDE pair can be formed with a MinD having an amino acid sequence selected from the group consisting of SEQ ID NOs: 54, 55, 58, 59, 62, 64, 65, and 66.

Focusing specifically on the MinD having an amino acid sequence including SEQ ID NO: 60, a functioning MinDE pair can be formed with a MinE having an amino acid sequence of SEQ ID NO: 78. Focusing specifically on the MinE having an amino acid sequence including SEQ ID NO: 73, no functioning MinDE pair can be formed using the MinD that were sampled.

Focusing specifically on the MinD having an amino acid sequence including SEQ ID NO: 61, a functioning MinDE pair can be formed with a MinE having an amino acid sequence of SEQ ID NO: 74. Focusing specifically on the MinE having an amino acid sequence including SEQ ID NO: 74, a functioning MinDE pair can be formed with a MinD having an amino acid sequence selected from the group consisting of SEQ ID NOs: 54, 55, 58, 59, 62, 64, 65, and 66. It should be appreciated that, at least for the sampled MinD and MinE, the MinDE pair from Helicobacter pylori is orthogonal to all of the other sampled MinDE pairs. In other words, for the sampled MinDE pairs, the MinDE pair from H. pylori is effectively a universally orthogonal MinDE pair.

Focusing specifically on the MinD having an amino acid sequence including SEQ ID NO: 62, a functioning MinDE pair can be formed with a MinE having an amino acid sequence selected from the group consisting of SEQ ID NOs: 67, 68, 69, 70, 71, 72, 75, 76, 77, 78, and 79. Focusing specifically on the MinE having an amino acid sequence including SEQ ID NO: 75, a functioning MinDE pair can be formed with a MinD having an amino acid sequence selected from the group consisting of SEQ ID NOs: 54, 55, 59, 62, 64, 65, and 66.

Focusing specifically on the MinD having an amino acid sequence including SEQ ID NO: 63, no functioning MinDe pair can be formed using the MinE that were sampled. Focusing specifically on the MinE having an amino acid sequence including SEQ ID NO: 76, a functioning MinDE pair can be formed with a MinD having an amino acid sequence selected from the group consisting of SEQ ID NOs: 55, 62, 64, and 66.

Focusing specifically on the MinD having an amino acid sequence including SEQ ID NO: 64, a functioning MinDE pair can be formed with a MinE having an amino acid sequence selected from the group consisting of SEQ ID NOs: 67, 68, 69, 70, 72, 75, 76, 77, 78, and 79. Focusing specifically on the MinE having an amino acid sequence including SEQ ID NO: 77, a functioning MinDE pair can be formed with a MinD having an amino acid sequence selected from the group consisting of SEQ ID NOs: 54, 55, 62, 64, 65, and 66.

Focusing specifically on the MinD having an amino acid sequence including SEQ ID NO: 65, a functioning MinDE pair can be formed with a MinE having an amino acid sequence selected from the group consisting of SEQ ID NOs: 67, 68, 69, 70, 71, 72, 75, 77, 78, and 79. Focusing specifically on the MinE having an amino acid sequence including SEQ ID NO: 78, a functioning MinDE pair can be formed with a MinD having an amino acid sequence selected from the group consisting of SEQ ID NOs: 54, 55, 58, 59, 60, 62, 64, 65, and 66.

Focusing specifically on the MinD having an amino acid sequence including SEQ ID NO: 66, a functioning MinDE pair can be formed with a MinE having an amino acid sequence selected from the group consisting of SEQ ID NOs: 67, 68, 70, 71, 72, 75, 76, 77, 78, and 79. Focusing specifically on the MinE having an amino acid sequence including SEQ ID NO: 67, a functioning MinDE pair can be formed with a MinD having an amino acid sequence selected from the group consisting of SEQ ID NOs: 54, 55, 58, 59, 62, 64, 65, and 66.

In some embodiments the spatial and temporal distribution of molecules occurs within a cell. The cell may be a eukaryotic cell including a mammalian cell. By way of example, but not limitation the cell may be a U2OS, K562, 293T, 3T3, Jurkat, or MDCK cell. Cells may be a stable cell line or immortalized cell, a self renewing cell or a primary cell. A primary cell is one that is directly isolated from the parental tissue.

In some embodiments, the inventors contacted the cells with polynucleotides or nucleic acids encoding one or more components of the MinDE system, such that the polynucleotides were delivered to the cells by transfection. By way of example, and not limitation, these methods include: chemical transfection such as with calcium phosphate, synthetic lipid-based reagents such as cationic lipids, nonliposomal reagents, or physical transfection including electroporation, microinjection, or biolistic particle delivery. Transfections may be transient or stable.

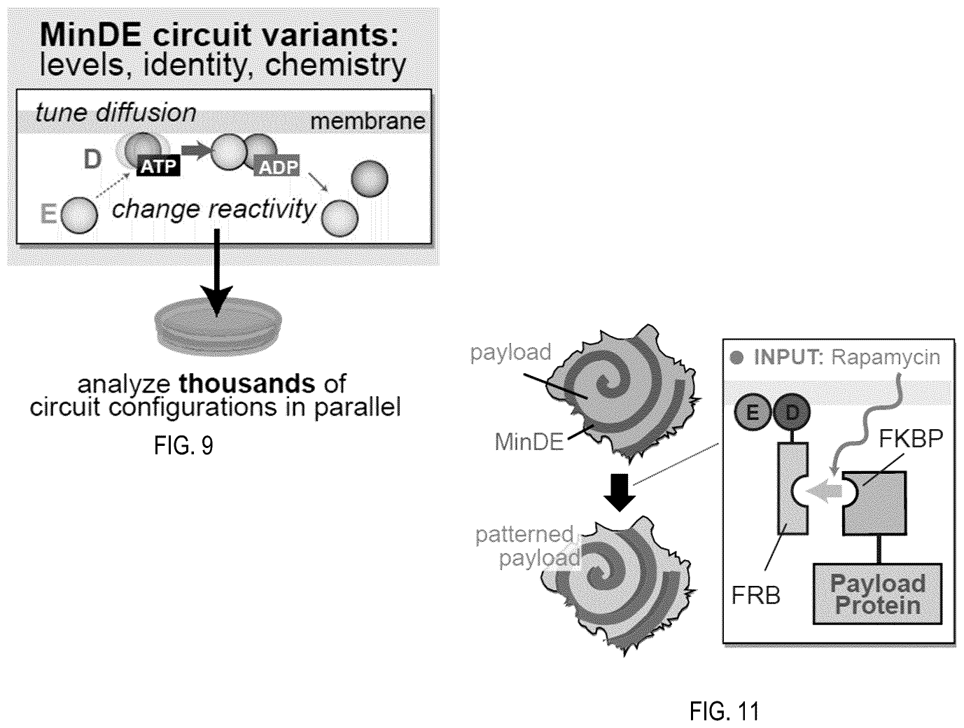

The inventors have demonstrated that the absolute and relative expression levels of MinD and MinE set the frequency and amplitude of the oscillation (see FIGS. 9-10) any synthetic biology strategy that precisely sets these gene expression levels in a given cell can be used. Example strategies may include, 1) Transduction of cells with separate MinD and MinE viruses and using FACS sorting to select populations with specific levels of interest, or 2) more hard-wired control can be obtained by using specific DNA sequences in which different promoters strengths, degron sequences, and copy numbers can be used to tune the levels of E and D in the cell. These techniques are known in the art.

Without wishing to be bound by any particular theory, since it has been demonstrated that the absolute and relative expression levels of MinD and MinE set the frequency and amplitude of the oscillations, a synthetic biology strategy that precisely sets these gene expression levels in a given cell can be used with the methods described herein to produce desired oscillation patterns. Examples of suitable strategies include the following: 1) transduction of cells with separate MinD and MinE viruses and using FACS sorting to select populations with specific levels of interest; and more hard-wired control obtained by using specific DNA sequences in which different promoters strengths, degron sequences, and copy numbers can be used to tune the levels of E and D in the cell. Without wishing to be bound by any particular theory, it is believed that the same general scaling relationships will be applicable to different cell types, though the precise relationships may vary.

In some embodiments, the spatiotemporal dynamics create patterns within the cell. By way of example and not limitation, these patterns may include directing whole-cell oscillations, traveling waves, spirals, turbulent patterns or static patterns. In some cases, the spatiotemporal dynamics create characteristic frequencies, which can be utilized in systems and methods described herein.

In some embodiments, the method comprises directing spatial and temporal distribution of a cargo molecule within a cell. A cargo molecule may be any molecule for which directing the spatial and temporal distribution within a cell is desired. Cargo may comprise proteins, RNA, DNA small molecules or lipids. By way of example, and not limitation, protein cargo may comprise condensates, microtubules, actin or signaling proteins. Cargo may also comprise RNA and or RNA and protein, including M2S protein/M2S RNA hairpin interaction or U1A binding protein/U1A RNA hairpin. DNA cargo may comprise binding domains or a CRISPR/Cas9-gRNA.

The present disclosure also provides methods. One method is a method of making and/or using the cells or compositions described herein. Another method is a method of analyzing images to take advantage of the spatial and temporal distributions of molecules that can be achieved as a result.

With respect to the method of making the cells or compositions described herein, a skilled artisan will recognize there are a variety of ways for expressing the MinDE system in the Eukaryotic cells described herein. This disclosure is not intended to be limiting.

With respect to one method of using the cells or compositions described herein, a skilled artisan will recognize that simply allowing the cells to live in appropriate conditions provides the functionality of the MinDE system, so the method can simply be providing proper conditions for the cells to exist.

In some cases, the functional moiety is not the end-target for coupling to the spatial and temporal distribution within a cell and the actual end-target is a peripheral molecule that needs to be bound to the functional moiety by way of an activating agent. In these cases, the “signal” that is desired for coupling to the spatial and temporal distribution is turned “off” when the activating agent is absent and gets turned “on” when the activating agent is added. This is illustrated herein with rapamycin mediated induction of a PPI (Example 1). Other possibilities include, light (using an optogenetic interaction pair); for example a Lov domain and its associated light-dependent interaction peptide; or Phy/Pif, other chemically induced dimerizes (Auxin interaction-domain) or reverse-dimerizes (developed by Takara) or induction of a competitor to peel it off—that is, interactions can be held in place by a low-affinity leucine zipper and displaced by inducible expression (through a TetON system) of a higher affinity leucine zipper interaction.

With respect to another method of using the compositions described herein, the ability to provide cells with a characteristic frequency can be used in a specific method of tracking cells. This method involved acquiring images of a population of cells. The images need to be acquired at a repetition rate that allows the various characteristic frequencies to be observed (i.e., no undersampling). Using techniques known in the art, such as lock-in amplification, the images can be analyzed to identify cells based on characteristic frequencies, which allows identification and tracking of a target cell. This technique overcomes many of the shortcomings of previous methods, because this characteristic frequency does not become modulated by virtue of a cluttered image scene, including other cells overlapping and potentially covering the target cell. In the case of this method, so long as cells are not entirely opaque, the frequency of a target cell should transmit through a second cell, so that the methods described herein can still track the target cell, despite it being covered by the second cell. When compared with traditional labeling techniques, such as labeling with unique fluorescent markers, this technique significantly extends the tracking capabilities into a third dimension, which was not previously accessible using existing techniques.

Another aspect of the present disclosure provides a method of directing the spatial and temporal distribution of molecules within a cell. The method comprising non-natively expressing a pair of proteins within a cell, wherein the pair of proteins exhibits whole-cell oscillations, traveling waves, spirals, turbulent patterns, static patterns, or combinations thereof. Expressing non-native proteins comprises any means known in the art for protein expression including those disclosed herein. Non-native proteins include those which are not naturally occurring within the cell from which they are being expressed. By way of example, and not limitation these include any proteins in the bacterial Min system, as well as orthologous systems in other species. Other orthologous systems include but are not limited to the members of ATPase/ATPase-activator system pairs including ParA which use substrates other than membranes to support oscillation. Further, other natural systems, unrelated to the Min system, but comprise similar spatiotemporal action within a cell, including, but not limited to GTPases, NTPase/NTPases activation systems and synthetic proteins may be used as described herein.

Another aspect of the present disclosure provides a method of exploiting spatial and temporal distribution behaviors generated by non-natively expressed proteins pairs. The method comprises, a) observing a spatial and temporal distribution of molecules within a cell including whole-cell oscillations, traveling waves, spirals, turbulent patterns, static patterns, or combinations thereof being generated by a pair of proteins with a cell, wherein the pair of proteins is not natively expressed in the cell, and b) expressing the pair of proteins within the cell, wherein a functional moiety is coupled to one of the pair of proteins, thereby coupling the functional moiety to the spatial and temporal distribution of molecules within the cell. A functional moiety comprises any such molecule which provides activity or purpose within the cell. By way of example, and not limitation, functional moieties may include mechanisms for, payload delivery, amplification of biochemical signals or frequency-modulation. Function moieties may further comprise transport, signaling, localization, cell growth, division or replication, metabolism or energy production or utilization molecules. Functional moieties may further comprise those which react to a report cost-cell signaling activities as well as those which self-organize host-cell activities to generate new cell behaviors.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The present disclosure teaches the successful development and implementation of a minimal protein circuit that can be programmed to produce waves, oscillations, and geometric patterns in human cells. The fast oscillatory dynamics achievable with this circuit are unprecedented in synthetic biology.

### Example 1

We sought a new paradigm: genetically encoded circuits that synthetically direct the spatiotemporal organization of proteins within a cell. To achieve this, we repurposed a positioning circuit from bacteria—the MinDE system—which is orthogonal to Eukaryotes. In E. coli cells, the division machinery is localized by pole-to-pole protein oscillations on the plasma membrane driven by a reaction-diffusion process: nucleotide-dependent membrane association of the MinD ATPase is antagonized by its ATPase-activating protein MinE. In vitro, MinD and MinE are sufficient to drive dynamic protein behaviors on supported lipid bilayers. These minimal requirements suggested that MinDE might produce reaction-diffusion behaviors in mammalian cells, providing a starting point for more complex spatiotemporal circuit design (FIG. 2).

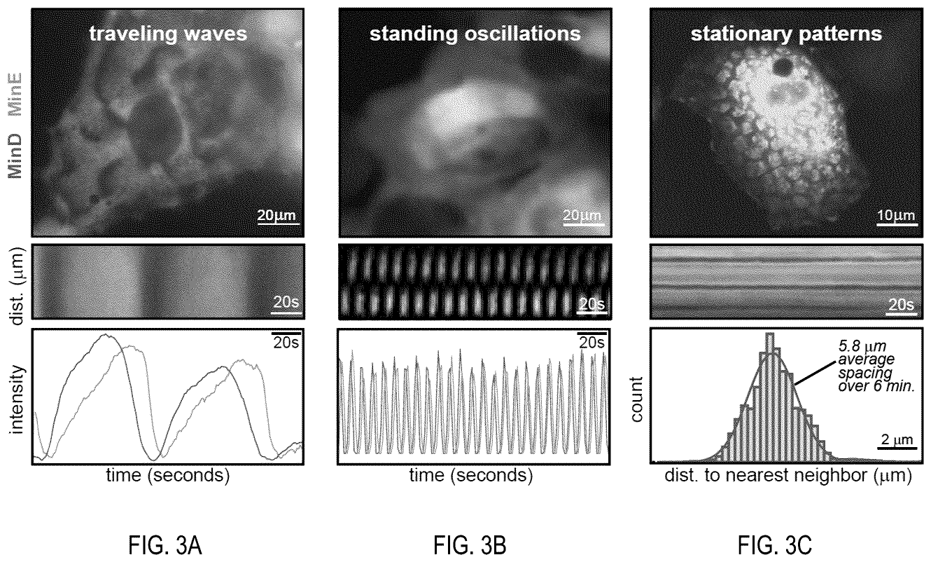

We used lentivirus to express mCherry-MinD and MinE-GFP in human cell lines and imaged their spatiotemporal distribution by timelapse fluorescence microscopy. Across a population of cells harboring different MinD and MinE expression levels, we observed a stunning array of self-organizing protein dynamics and patterning occurring within single cells (FIGS. 3A-3C, see also, portions of U.S. 63/425,294 and movies that accompany R. Rajasekaran et al., “A programmable reaction-diffusion system for spatiotemporal cell signaling circuit design,” bioRxiv 2022.11.15.516470; doi: https://doi.org/10.1101/2022.11.15.516470, hereinafter “Rajasekaran et al.”, which are incorporated by reference herein in their entirety). This included traveling waves, spirals, and turbulent patterns with periods ranging from seconds to minutes (FIG. 3A and portions of U.S. 63/425,294); fast standing oscillations with persistent nodal structure (FIG. 3B and portions of U.S. 63/425,294); and stationary Turing-type “leopard print” patterns in which regularly spaced protein domains maintained their position over time (FIG. 3C and portions of U.S. 63/425,294).

These phenomena could be produced in multiple different adherent and suspension cell-lines (See, portions of U.S. 63/425,294 and movies that accompany Rajasekaran et al.). In all cases, the MinDE patterns were distributed throughout the entire cell body, and confocal imaging suggested this whole-cell patterning was supported by MinDE interaction with the endomembrane system of the cell (see portions of US 63/425,294 and movies that accompany Rajasekaran et al.). Detailed inspection of MinD and MinE fluorescence signals over time showed that for dynamic patterns, MinE slightly lagged MinD (FIG. 3A-3B and portions of U.S. 63/425,294); and for stationary patterns, domains of MinD were encircled at their perimeter by a ring of MinE (FIG. 3C and portions of U.S. 63/425,294). This is the expected spatiotemporal organization of a two-component reaction-diffusion system and agrees with past in vitro observations of reconstituted MinDE systems.

This genetically encoded protein patterning system is powerful because its behavior will be amenable to tuning and control using synthetic biology and protein engineering strategies. Moreover, the resulting protein patterns can serve as the foundation for more elaborate signaling circuits that react to or control endogenous activities or pathways in the host cell. As such, we explored how MinDE circuits and the signals they generate could be analyzed, engineered, and controlled for useful applications.

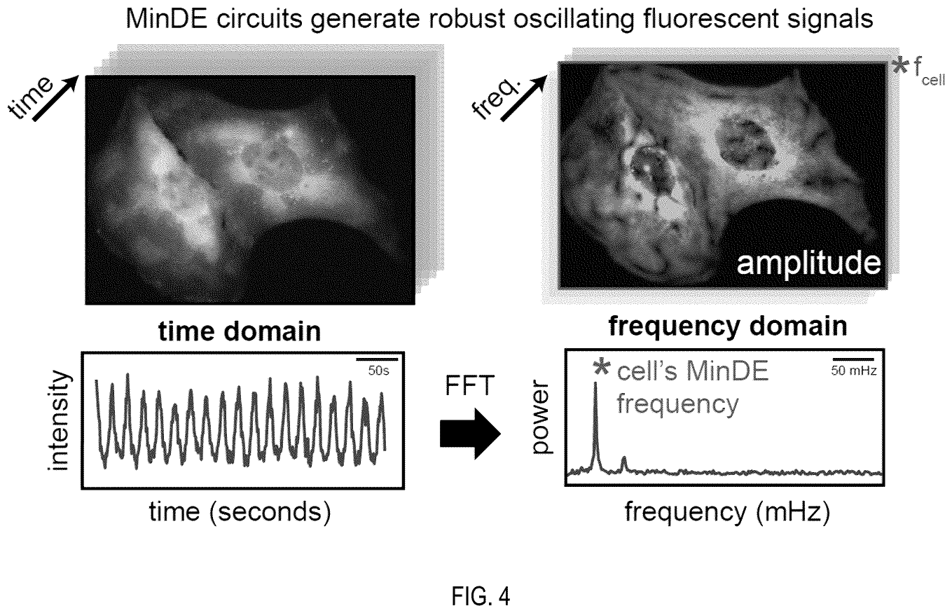

To analyze the content of MinDE signals quantitatively, we took advantage of the fact that the pixel-level MinDE fluorescence dynamics at any point in the cell were strongly oscillatory (FIGS. 4, 9, and 10 and portions of U.S. 63/425,294). Using Fast-Fourier Transform (FFT), we mapped these pixel-level dynamics to the frequency-domain. The pixel-level power spectrum showed a strong peak associated with the frequency of the MinDE oscillation at that location (FIG. 4). These pixel-level FFTs were used to produce an “image power spectrum”, in which each slice in the image-stack corresponds to a different frequency and pixel intensity corresponds to the oscillation power of that frequency (FIG. 4). In these stacks, an individual cell is located at a particular frequency slice, indicating that a MinDE circuit generates a specific cell-wide temporal frequency. The associated phase angle of the oscillation for each pixel in that frequency slice (the phase field) provides additional data about the underlying spatial structure of that cell's MinDE circuit, including its wavelength, defect distribution, and nodal structure (see portions of U.S. 63/425,294 and movies that accompany Rajasekaran et al.).

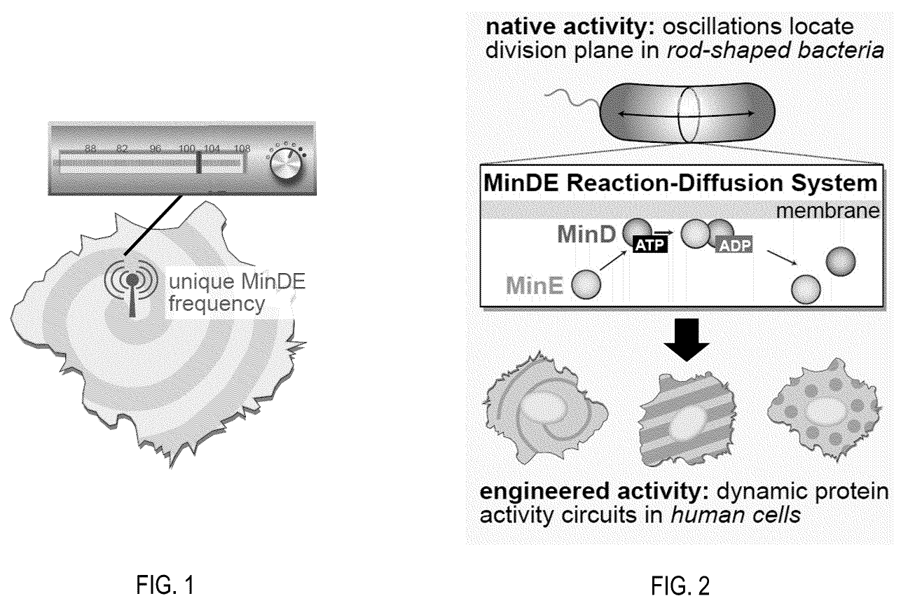

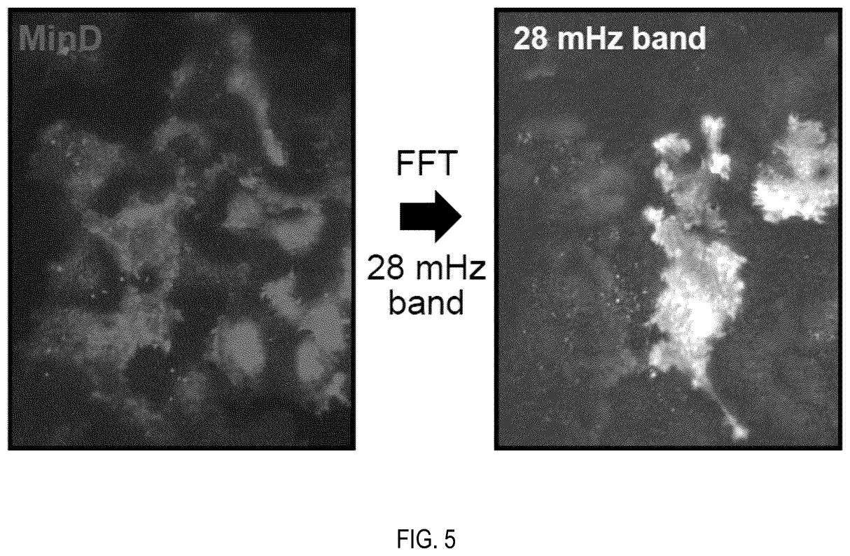

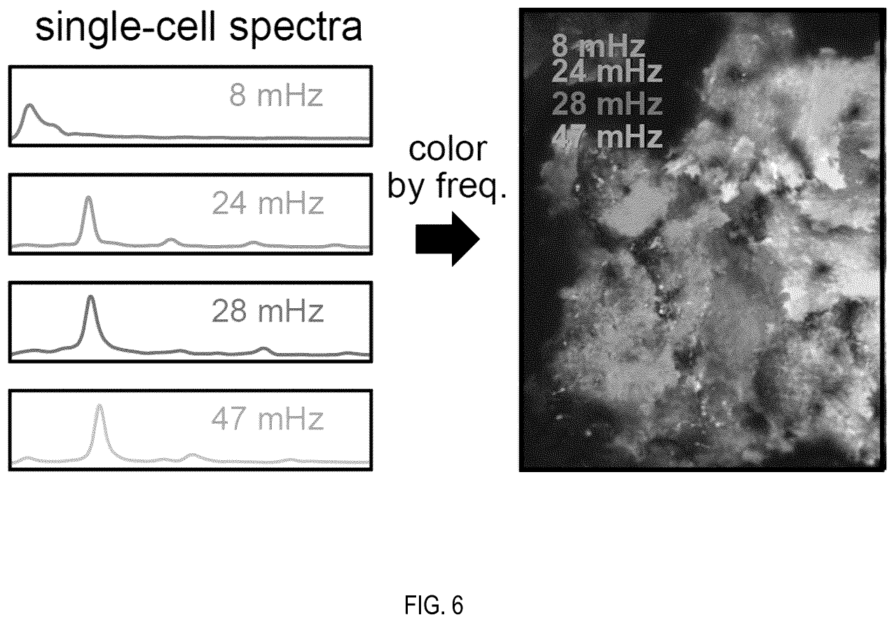

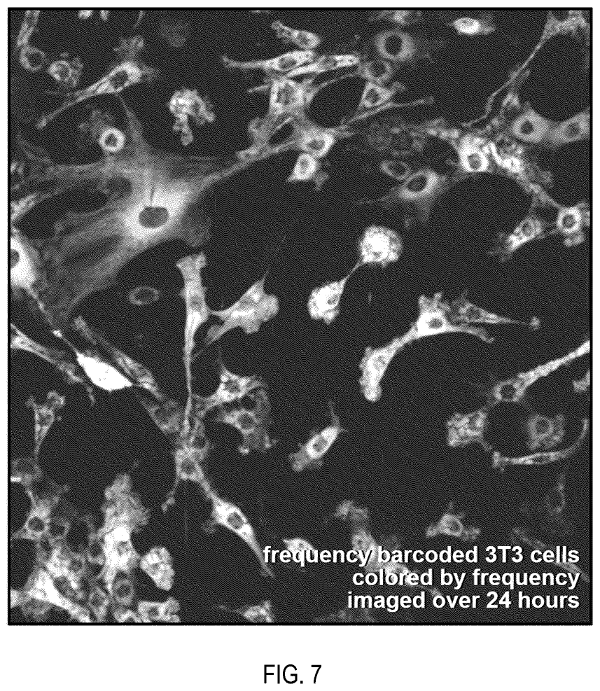

Given that the temporal frequency of a cell's MinDE signal is uniform, it provides a frequency-domain single-cell imaging barcode that acts like a “cellular radio station” (FIG. 1). This surprising fact enabled us to perform novel filtering, isolation, and analysis of MinDE fluorescence signals using techniques from digital signal processing. Fluorescence from different cells within a mixed population could be isolated by simply tuning to different slices in the image power spectrum. Thus, locating and segmenting cell boundaries becomes trivial even at elaborate cell-cell interfaces (FIG. 5 and portions of U.S. 63/425,294). Moreover, signals from physically overlapping cells with distinct MinDE frequencies could be decomposed and assigned back to their cell of origin (FIGS. 6 & 7). For example, within a population of migrating 3T3 cells, these frequency-barcodes could label and track single cells even as they interacted and overlapped with one another. This enabled us to produce a multi-channel rendering of the time series data in which individual cells were colored by frequency, using only a single fluorophore (FIG. 7 and movies that accompany Rajasekaran et al.).

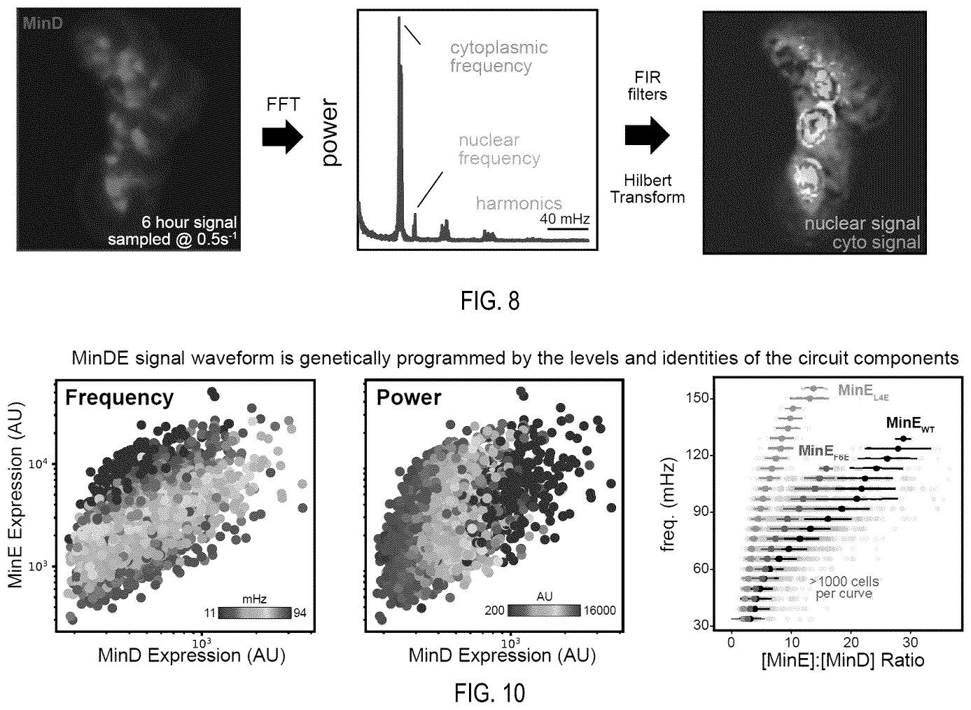

We also found that we could separate MinDE signals arising from different sub-cellular compartments within the same cell. In a 6-hour recording of a U2OS cell, the associated power spectrum showed peaks at the fundamental frequency of the MinDE oscillation in the cell body and its harmonics, as well as an additional small peak corresponding to a weak secondary MinDE oscillation in the nucleus (FIG. 8). We separated the nuclear and cytoplasmic signals from within this single-channel recording using finite-impulse-response (FIR) filters. We then recovered the instantaneous power, phase, and frequency of the resulting signals using the Hilbert Transform, generating a multi-channel rendering that labeled each sub-cellular compartment (FIG. 9 and portions of U.S. 63/425,294 and movies that accompany Rajasekaran et al.). Nearly identical results were obtained using continuous wavelet transform (see portions of U.S. 63/425,294 and movies that accompany Rajasekaran et al. Rajasekaran et al.). Thus, MinDE signals provide an unprecedented means of encoding and decoding single-cell and sub-cellular data in synthetic protein dynamics that can be analyzed and recovered using a suite of tools from digital signal processing.

Given the utility of the signals MinDE circuits generate, we sought to program their dynamic behavior at genetic level. We imaged thousands of MinDE circuit configurations spanning a wide range of MinD-mCherry and MinE-GFP expression levels, segmented individuals based on their frequency, and extracted the average GFP and mCherry intensity within the cell as a proxy for MinE and MinD expression, respectively (FIG. 9). Aggregating these data, we found that MinDE signal frequency was strongly determined by the relative levels of MinD and MinE: for a fixed MinD expression level, higher MinE levels led to higher frequencies (FIG. 10). In contrast, the power (amplitude) of the MinDE signal was set by the MinD expression levels (FIG. 10). These trends were consistent across all cell lines we analyzed (see portions of U.S. 63/425,294). Thus, a specific MinDE signal frequency and amplitude can be genetically-encoded by controlling gene-expression levels.

These observations agree with the biochemical activities of MinD and MinE: MinD is the ATPase in the system, setting the maximum number of molecules that can participate in the oscillation; and MinE is the ATPase activator, whose concentration will determine how rapidly ATP is hydrolyzed by MinD. Consistent with this, mutant forms of MinE with faster membrane exchange produced faster oscillations (>160 mHz) and showed different frequency-scaling relationships (FIG. 10). This indicates that targeted mutations in MinDE components can be used to alter the mapping between expression levels are circuit dynamics.

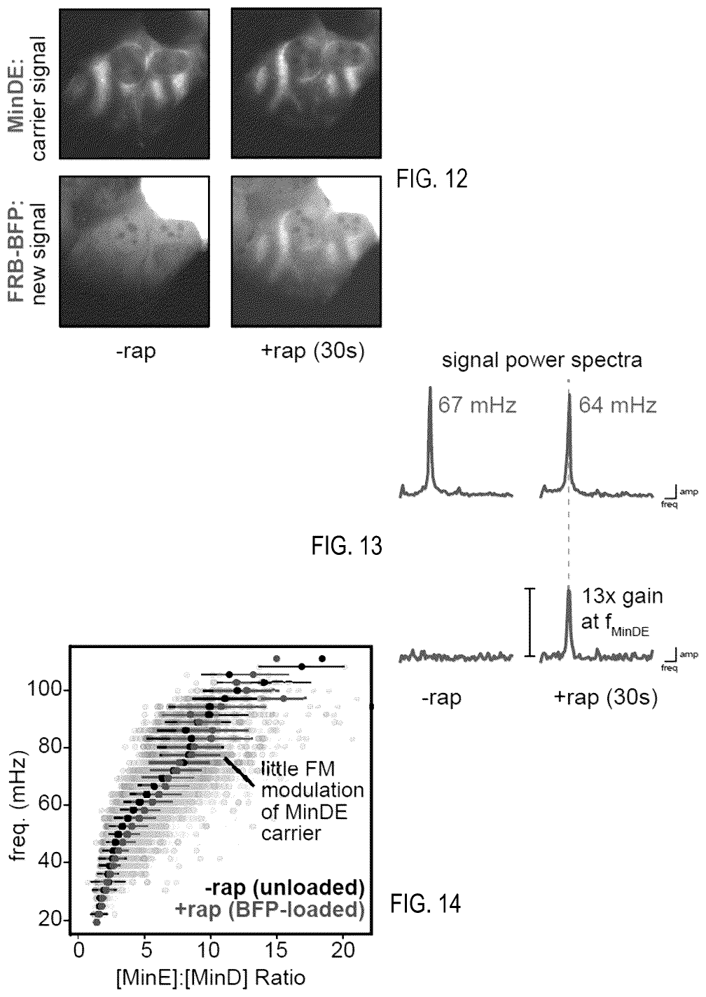

Because MinDE signals are fast and protein-based, we wondered whether more complex signaling dynamics or composite signals could be created by coupling MinDE to other proteins or cellular structures. To this end, we created a plug-and-play platform that dynamically connects MinDE to other components, based on the chemically inducible heterodimerization system FKBP/FRB (FIG. 11). When FRB was fused to MinD, an FKBP-BFP test payload did not colocalize with MinDE in the absence of rapamycin. However, within seconds of adding rapamycin, the BFP signal colocalized with MinDE in space and time (FIG. 12 and movies that accompany Rajasekaran et al.). As a result, this circuit generates a new oscillatory signal in the BFP channel derived from the MinDE carrier signal whose amplitude is modulated in real time by the FKBP/FRB interaction (FIG. 13).

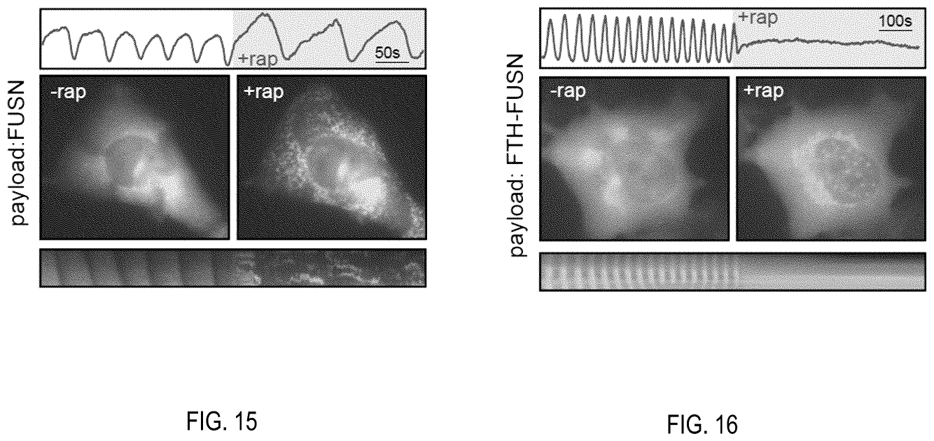

As MinDE interacts with other proteins in the cell, the spatiotemporal dynamics of the MinDE signal itself could also change in response. For a simple BFP payload, we observed little change in the steady-state frequency of the MinDE signal upon loading (FIG. 14); that is, the derived BFP signal largely inherits the frequency of the MinDE carrier it arises from. We then tested the effects of connecting MinDE to well-characterized “intrinsically disordered region” sequences (IDRs) with different propensities to form condensates, gels, or aggregate structures with altered diffusivity. While weak IDRs like RGG had little effect on MinDE dynamics (see portions of U.S. 63/425,294 and movies that accompany Rajasekaran et al.), recruitment of stronger IDRs derived from FUSN or DDX4 rapidly altered MinDE spatiotemporal behavior (FIG. 15 and portions of U.S. 63/425,294 and movies that accompany Rajasekaran et al.). For these circuits, we observed oscillating puncta assembly and disassembly along the wave trajectory, coincident with a decrease in the circuit frequency and redistribution of circuit power. In contrast, connection of MinDE circuits to large, preformed protein-condensates (via FTH1-FUSN) or to the microtubule cytoskeleton (via TPPP) led to immediate arrest of circuit oscillation (FIG. 16 and portions of U.S. 63/425,294 and movies that accompany Rajasekaran et al.).

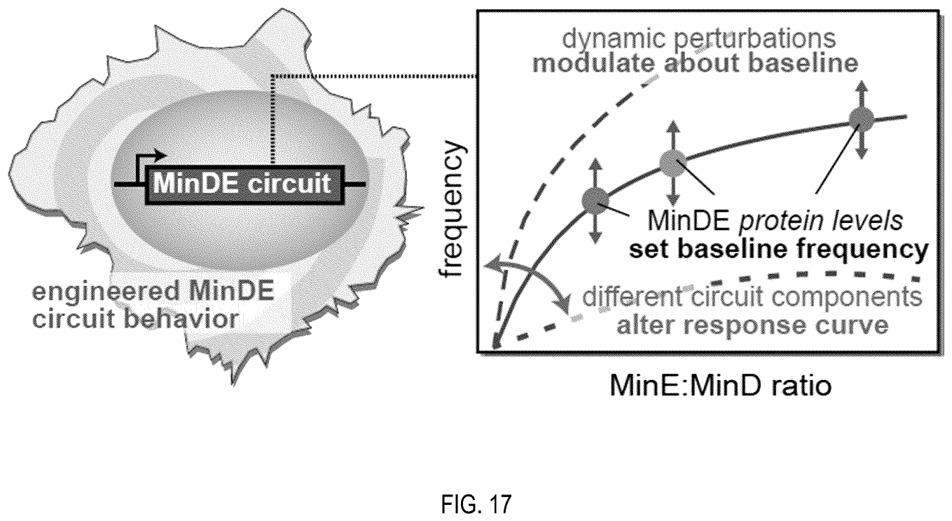

Taken together, these results indicate that the baseline MinDE spatiotemporal dynamics can be programmed at a genetic level and further modulated in real-time at the protein level through dynamic connection to other cellular components (FIG. 17). Depending on the nature of these interactions, MinDE dynamics can persist and drive other processes (such as condensate assembly and disassembly) or be overridden by stronger localization signals. Thus, the space of possibilities for sculpting MinDE behavior in living cells is vast, creating a new frontier for synthetically exploring reaction-diffusion dynamics, patterning, and signal generation.

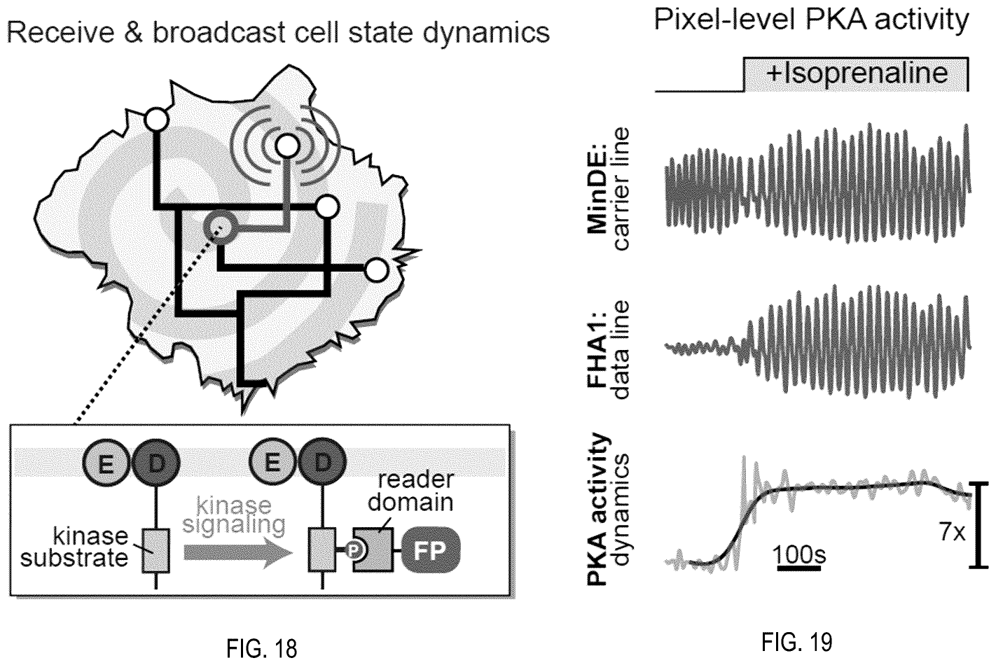

We next applied this knowledge to design specific MinDE circuits with novel applications for understanding or engineering cell biology. Taking advantage of the “radio” like properties of MinDE signals, we first created circuits that read out and broadcast frequency-barcoded dynamic cell-state data, using PKA kinase signaling activity as a test-case (FIG. 18). For this circuit, we fused a substrate sequence for PKA to MinD and co-expressed this with mCerulean-FHA, which specifically binds to the phosphorylated substrate sequence. Such substrate/reader pairs have previously been used to develop FRET-based or condensate-based activity reporters.

The resulting circuit generates a new signal derived from MinDE in the mCerulean channel whose amplitude is dynamically modulated by PKA signaling activity. In resting cells, this PKA-activity signal had low power as there was little colocalization of FHA-mCerulean with MinDE (FIG. 19 and portions of U.S. 63/425,294). However, upon stimulation with the PKA agonist isoprenaline, FHA immediately began co-oscillating with the MinDE carrier, leading to increase in the power of the PKA-activity signal (FIG. 19 and portions of U.S. 63/425,294). Importantly, the ratio of power between the MinDE carrier signal and the PKA activity signal was consistent everywhere within the cell, facilitating normalization, comparison and aggregation of the single-pixel signaling trajectories (FIG. 19 and portions of U.S. 63/425,294 and movies that accompany Rajasekaran et al.). For a saturating dose of isoprenaline, the increase in this power-ratio could be as much as seven-fold, providing outstanding signal to noise that compares favorably to FRET, translocation, or condensate-based reporter systems.

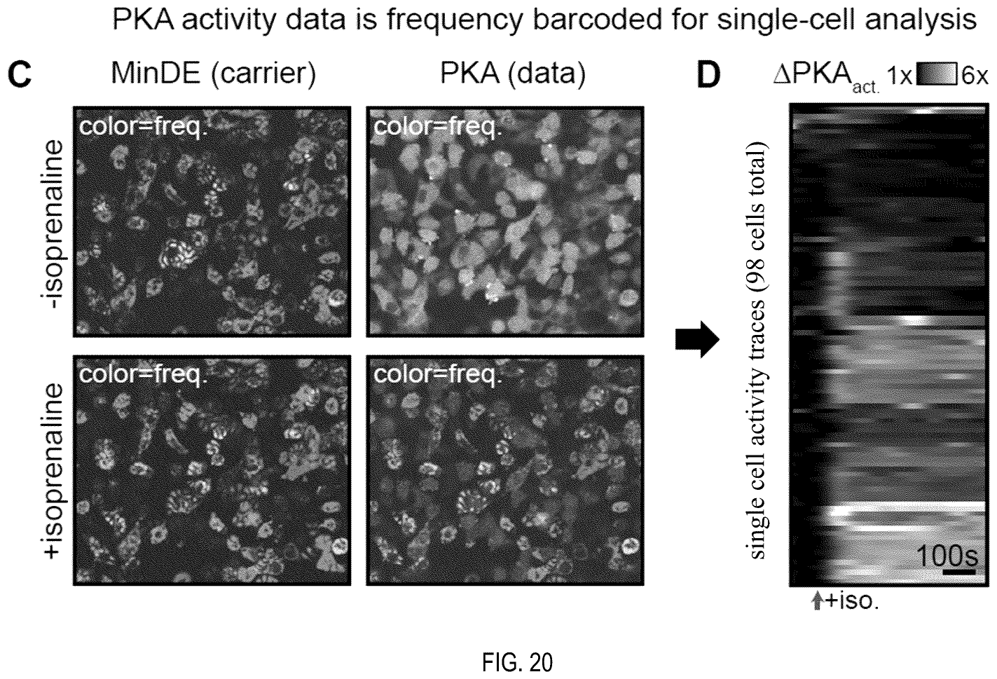

Critically, this readout of PKA signaling is barcoded by the cell's underlying MinDE frequency (FIG. 20). Thus, even when different cells physically overlap, the signaling activity of each individual cell can be spectrally isolated in the frequency-domain. This allowed rapid, high-throughput extraction of single-cell PKA signaling trajectories from fields of cells even at low magnification (FIG. 20). This ability to couple cellular data to the MinDE FM-barcode is not possible with any other imaging-based barcoding scheme. Moreover, because only on a single fluorophore is needed to track the MinDE carrier, it is possible to barcode and broadcast multiple data lines for different signaling pathways or activity reporters in parallel using this approach. To our knowledge, there are no existing strategies that provide the barcoding, amplification, and multiplexing potential of these MinDE cellular radio circuits.

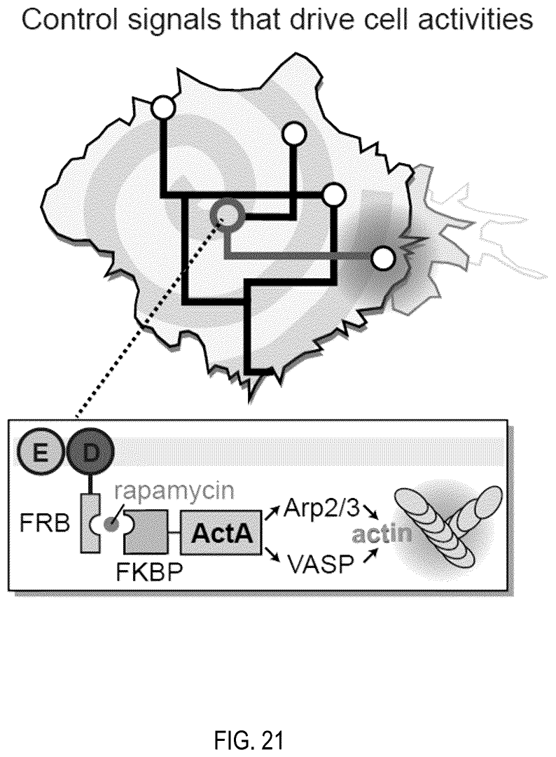

We then explored whether the same MinDE signals we used to broadcast endogenous cellular activities could also be used to synthetically reorganize them. To explore this, we built circuits that targeted a pathway that would produce an output signal clearly localized in space and time: actin polymerization. Many endogenous signaling pathways and bacterial effector proteins organize actin polymerization in the cell through signaling to the Arp2/3 complex. A fragment of one such effector from Listeria, ActA, can support synthetic actin polymerization when clustered on membrane surfaces. Using our plug-and-play inducible system, we generated cell lines in which an ActA payload could be coupled to MinDE spatiotemporal patterns and used LifeAct-GFP to monitor the associated actin dynamics in real-time (FIG. 21). This allowed us to query a range of MinDE-to-ActA control signals for their ability to support and organize actin polymerization.

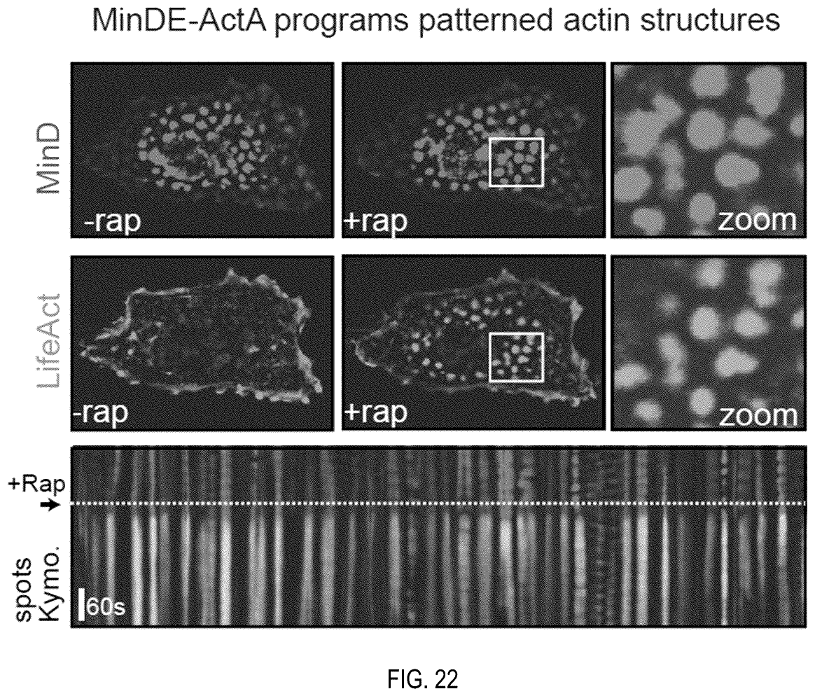

In cells displaying stationary MinDE patterns of geometric spots, the LifeAct-GFP signal did not colocalize with MinDE patterns in the absence of rapamycin. However, upon addition of rapamycin, we observed rapid production of filamentous actin structures with size, shape and position controlled by the associated MinDE pattern (FIG. 22 and portions of U.S. 63/425,294 and movies that accompany Rajasekaran et al.). This shows that the spatiotemporal organization encoded by a MinDE circuit can be used as a blueprint to productively localize host-cell signaling activities in space and time.

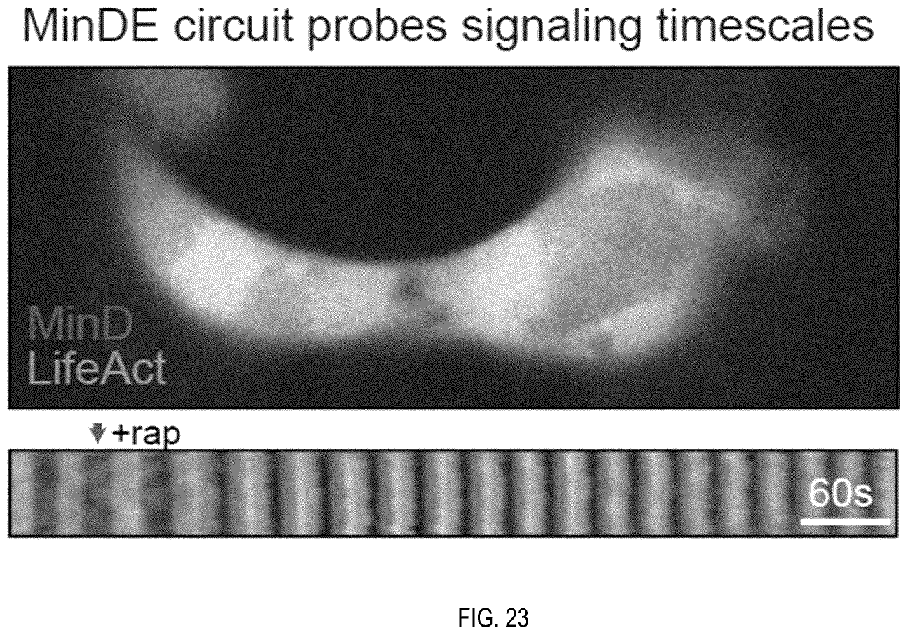

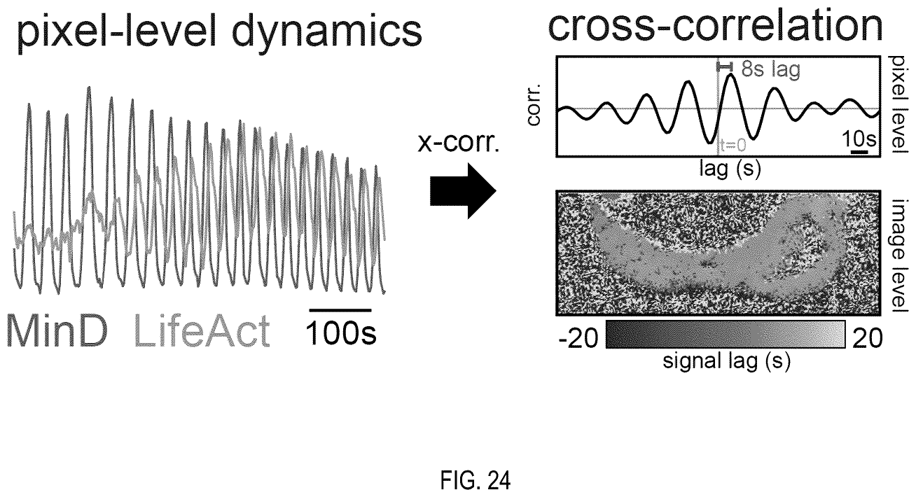

Interestingly, when ActA was recruited to dynamic MinDE patterns, LifeAct-GFP oscillated at the same frequency as MinDE but with a noticeable delay between the two signals (FIG. 23-24 and portions of U.S. 63/425,294 and movies that accompany Rajasekaran et al.). Cross-correlation of the oscillatory LifeAct and MinDE signals defined a time-lag of 6-10 seconds in the cells we inspected (FIG. 24 and portions of U.S. 63/425,294 and movies that accompany Rajasekaran et al.), which we interpret as the time it takes for MinDE recruitment of ActA to drive productive signaling to the actin polymerization machinery in our circuit context. We emphasize that this specific timescale likely depends on the expression levels of ActA, the power of the MinDE oscillation, and the availability of actin regulatory components in any individual cell. Nevertheless, it demonstrates that MinDE signals not only can act as genetically encoded spatiotemporal controllers of the cell, but can also dynamically probe spatiotemporal signaling constraints on the pathways that we connect them to.

We have shown that a bacterial reaction-diffusion system, MinDE, can be used as an orthogonal synthetic biology platform to generate self-organizing genetically-encoded protein oscillations, patterns, and spatiotemporal signaling circuits in mammalian cells. The oscillatory nature of MinDE signals enables them to be deployed as cellular radios that barcode single-cell identity or compartments with a unique frequency. By capitalizing on these novel properties, we developed specific circuits that can read out and control mammalian cell biology. Our work provides a foundation for building more complex protein-based reaction-diffusion circuits that incorporate feedback and sense data into their spatiotemporal dynamics, providing a new suite of tools for visualizing, probing, and perturbing cell biology. It also provides an experimental test-bed for leveraging synthetic biology to more generally explore the physics and chemistry of reaction-diffusion systems that incorporate time-varying protein-levels, localization, or diffusivity. Taken together, we establish a new paradigm for synthetic interaction with cells at length and timescales common to biology but traditionally difficult to program experimentally.

### Example 2

Cross-reactivity between MinD and MinE was investigated by transfecting equivalent amounts of plasmid DNA encoding fluorescently tagged versions of the proteins into HEK293T cells. All tested combinations were imaged using the same imaging parameters described in Example 1, such as number of fields of views collected, interval time between frames, total duration of imaging, and exposure times for fluorescence. Frequency-based cell segmentation was applied to extract single cell data sets for all MinD and MinE combinations tested. A binary map was prepared by setting a threshold of a minimum of 20 observed cells within a particular data set. Combinations that passed the threshold were further analyzed to extract the median frequency and median power displayed across all cells observed within the respective population.

Cross-reactivity between MinD and MinE was assessed and the results are shown in FIGS. 25-27. In FIG. 25, a binary plot shows functioning (white squares) and non-functioning (black squares) MinDE pairs. In FIG. 26, the frequency for the functional pairs is illustrated. In FIG. 27, the power of each functional pair is shown. This data illustrates that selecting MinDE pairs could provide two or more independent circuitries within the same cell.

We claim:

1. A method of directing the spatial and temporal distribution of molecules within a cell, the method comprising: expressing a first MinDE pair comprising a first MinD and a first MinE in a Eukaryotic cell.

2. The method of claim 1, the method further comprising: expressing a second MinDE pair comprising a second MinD and a second MinE in the Eukaryotic cell.

3. The method of claim 2, wherein the first MinDE pair is orthogonal to the second MinDE pair.

4. The method of claim 1, wherein a reporter molecule is coupled to the MinD and/or the MinE, thereby providing at least partial visualization of the spatial and temporal distribution of molecules within the cell.

5. The method of claim 4, the method further comprising: imaging the cell, thereby at least partly visualizing the spatial and temporal distribution of molecules within the cell.

6. The method of claim 5, wherein the imaging is frequency-specific and allows identification of a cell by virtue of a characteristic frequency generated by the first MinDE pair therein.

7. The method of claim 1, wherein the spatial and temporal distribution of molecules within the cell includes whole-cell oscillations, traveling waves, spirals, turbulent patterns, static patterns, or combinations thereof.

8. The method of claim 1, wherein the Eukaryotic cell is a mammalian cell.

9. The method of claim 1, wherein a functional moiety is coupled to the first MinD and/or the first MinE, thereby coupling the functional moiety to the spatial and temporal distribution of molecules within the cell.

10. The method of claim 9, the method further comprising: adding an activating agent to the cell, thereby providing coupling between the functional moiety and a peripheral agent, thereby coupling the peripheral agent to the first MinD and/or the first MinE to which the functional moiety is coupled and coupling the peripheral agent to the spatial and temporal distribution of molecules within the cell.

11. The method of claim 10, wherein the activating agent is rapamycin.

12. The method of claim 1, wherein the first MinD or the first MinE has an amino acid sequence selected from the group consisting of SEQ ID NOs: 54-79 or a sequence having at least 95% sequence identity to SEQ ID NOs: 54-79.

13. The method of claim 1, wherein the first MinD has an amino acid sequence selected from the group consisting of SEQ ID NOs: 54, 55, 58, 59, 60, 61, 62, 64, 65, and 66 or a sequence having at least 95% sequence identity to SEQ ID NOs: 54, 55, 58, 59, 60, 61, 62, 64, 65, and 66.

14. The method of claim 1, wherein the first MinE has an amino acid sequence selected from the group consisting of SEQ ID NOs: 67, 68, 69, 70, 71, 72, 74, 75, 76, 77, 78, and 79 or a sequence having at least 95% sequence identity to SEQ ID NOs: 67, 68, 69, 70, 71, 72, 74, 75, 76, 77, 78, and 79.

15. A composition of matter comprising: a cell non-natively expressing a first MinDE pair comprising a first MinD and a first MinE.

16. The composition of claim 15, wherein the cell non-natively expresses a second MinDE pair comprising a second MinD and a second MinE.

17. The composition of claim 16, wherein the first MinDE pair is orthogonal to the second MinDE pair.

18. The composition of claim 15, wherein the first MinD has an amino acid sequence selected from the group consisting of SEQ ID NOs: 54, 55, 58, 59, 60, 61, 62, 64, 65, and 66 or a sequence having at least 95% sequence identity to SEQ ID NOs: 54, 55, 58, 59, 60, 61, 62, 64, 65, and 66.

19. The composition of claim 15, wherein the first MinE has an amino acid sequence selected from the group consisting of SEQ ID NOs: 67, 68, 69, 70, 71, 72, 74, 75, 76, 77, 78, and 79 or a sequence having at least 95% sequence identity to SEQ ID NOs: 67, 68, 69, 70, 71, 72, 74, 75, 76, 77, 78, and 79.

20. A method of exploiting spatial and temporal distribution behaviors generated by non-natively expressed proteins pairs, the method comprising:
a) observing a spatial and temporal distribution of molecules within a cell including whole-cell oscillations, traveling waves, spirals, turbulent patterns, static patterns, or combinations thereof being generated by a pair of proteins with a cell, wherein the pair of proteins is not natively expressed in the cell;
b) expressing the pair of proteins within the cell, wherein a functional moiety is coupled to one of the pair of proteins, thereby coupling the functional moiety to the spatial and temporal distribution of molecules within the cell.


SEQUENCE LISTING (79 sequences)

SEQ ID NO: 1 - DNA, 21 bp, synthetic construct
  source 1..21: mol_type = other DNA; note = pLV-EF1a-mCherry-MinD-IRES-Puro; Baseline Circuit; primer to SEQ ID 5; 3328..3348; EF1a-F
tcaagcctcagacagtggttc

SEQ ID NO: 2 - DNA, 49 bp, synthetic construct
  source 1..49: mol_type = other DNA; note = pLV-EF1a-mCherry-MinD-IRES-Puro; Baseline Circuit; primer to SEQ ID 5; 3356..3404; mCherry-MinD F
ttttcttccatttcaggtgtcgtgaggatccgccaccatggtgagcaag

SEQ ID NO: 3 - DNA, 49 bp, synthetic construct
  source 1..49: mol_type = other DNA; note = pLV-EF1a-mCherry-MinD-IRES-Puro; Baseline Circuit; primer to SEQ ID 5; complement(4929..4977); snap-MinD R
taccgcggcccgtcgactctagagcggccgcttatcccccgaacagccg

SEQ ID NO: 4 - DNA, 21 bp, synthetic construct
  source 1..21: mol_type = other DNA; note = pLV-EF1a-mCherry-MinD-IRES-Puro; Baseline Circuit; primer to SEQ ID 5; complement(4998..5018); pLV-EF1a-R
tagagggaaaccgttgctagc

SEQ ID NO: 5 - DNA, 10253 bp, unidentified
  source 1..10253: mol_type = other DNA; note = pLV-EF1a-mCherry-MinD-IRES-Puro; Baseline Circuit
  CDS 4134..4943: protein_id = 6; translation = MARIIVVTSGKGGVGKTTSSAAIATGLAQKGKKTVVIDFDIGLR NLDLIMGCERRVVYDFVNVIQGDATLNQALIKDKRTENLYILPASQTRDKDALTREGV AKVLDDLKAMDFEFIVCDSPAGIETGALMALYFADEAIITTNPEVSSVRDSDRILGIL ASKSRRAENGEEPIKEHLLLTRYNPGRVSRGDMLSMEDVLEILRIKLVGVIPEDQSVL RASNQGEPVILDINADAGKAYADTVERLLGEERPFRFIEEEKKGFLKRLFGG
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggacagcgccggaagtgctggctctgcgggtaccatggcccgcataatagttgtgacttcaggtaagggcggagttggaaaaaccacatccagcgcagccatagctaccggcctcgcacagaaaggcaagaaaactgtggtcatagattttgatattgggctcaggaacctggacctgataatgggctgcgaacggcgcgttgtctacgattttgtcaatgtcatccaaggcgacgcgactcttaatcaggcacttataaaagataaacgaaccgaaaacttgtatattcttcctgcctcacaaacgcgggacaaagacgcgctcaccagggagggtgtcgcaaaagttttggatgacttgaaagcgatggactttgagttcatagtttgtgactctcccgccggtatagagacaggggcacttatggcactgtacttcgcagatgaagccataatcacaactaatccggaggtgtctagtgtgcgagattcagaccgaatactggggatactcgccagcaaatcacggagggcagaaaacggggaggagccaattaaggagcatctcctgcttacccgatataaccccggtcgagtatctcggggagatatgctttcaatggaagatgtcctcgagattctgaggataaaactggtaggcgttatcccggaagaccagtccgtcctccgcgcatctaatcagggggagcctgtcatacttgacataaatgcggatgctgggaaagcttatgccgatacggtagaacggctcctcggggaagaacggcccttccgcttcatagaggaggaaaagaagggattccttaaacggctgttcgggggataagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 6 - protein, 270 aa, unidentified
  source 1..270: note = pLV-EF1a-mCherry-MinD-IRES-Puro; Baseline Circuit CDS
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 7 - DNA, 21 bp, synthetic construct
  source 1..21: mol_type = other DNA; note = pLV-EF1a-MinE-EGFP-IRES-Blast; Baseline Circuit; primer to SEQ ID 10 at 6565..6585 EF1a-F
tcaagcctcagacagtggttc

SEQ ID NO: 8 - DNA, 49 bp, synthetic construct
  source 1..49: mol_type = other DNA; note = pLV-EF1a-MinE-EGFP-IRES-Blast; Baseline Circuit; primer to SEQ ID 10 at 6593..6641 /label="MinE-clip F"
ttttcttccatttcaggtgtcgtgaggatccgccaccatggcccttctt

SEQ ID NO: 9 - DNA, 21 bp, synthetic construct
  source 1..21: mol_type = other DNA; note = pLV-EF1a-MinE-EGFP-IRES-Blast; Baseline Circuit primer to SEQ ID 10; complement(7698..7718) /label="pLV-EF1a-R"
tagagggaaaccgttgctagc

SEQ ID NO: 10 - DNA, 9545 bp, unidentified
  source 1..9545: mol_type = other DNA; note = pLV-EF1a-MinE-EGFP-IRES-Blast; Baseline Circuit
  CDS 6630..7646: protein_id = 11; translation = MALLDFFLSRKKNTANIAKERLQIIVAERRRSDAEPHYLPQLRK DILEVICKYVQIDPEMVTVQLEQKDGDISILELNVTLPEAEELKDSAGSAGSAGTMVS KGEELFTGVVPILVELDGDVNGHKFSVSGEGEGDATYGKLTLKFICTTGKLPVPWPTL VTTLTYGVQCFSRYPDHMKQHDFFKSAMPEGYVQERTIFFKDDGNYKTRAEVKFEGDT LVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYK
tggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggcccttcttgacttcttcctgtctcgaaaaaagaacacagcgaatatcgcgaaagaacggttgcaaattatagtggcggagagacgacgaagcgacgcggagcctcattaccttcctcaattgcgaaaagacatactggaagtgatctgcaagtacgttcaaattgaccctgagatggtgaccgtgcaacttgaacagaaggatggagatataagcatcctggaattgaacgtaacccttcccgaggcggaagaacttaaagacagcgccggaagtgctggctctgcgggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtcatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggac

SEQ ID NO: 11 - protein, 338 aa, unidentified
  source 1..338: note = pLV-EF1a-MinE-EGFP-IRES-Blast; Baseline Circuit CDS
MALLDFFLSRKKNTANIAKERLQIIVAERRRSDAEPHYLPQLRKDILEVICKYVQIDPEMVTVQLEQKDGDISILELNVTLPEAEELKDSAGSAGSAGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 12 - DNA, 21 bp, synthetic construct
  source 1..21: mol_type = other DNA; note = pLV-EF1a-MinE-F6E-EGFP-IRES-Blast; Baseline Circuit primer to SEQ ID 15 at 6565..6585 /label="EF1a-F"
tcaagcctcagacagtggttc

SEQ ID NO: 13 - DNA, 73 bp, synthetic construct
  source 1..73: mol_type = other DNA; note = pLV-EF1a-MinE-F6E-EGFP-IRES-Blast; Baseline Circuit primer to SEQ ID 15 at 6593..6665 /label="MinE_F6E"
ttttcttccatttcaggtgtcgtgaggatccgccaccatggcccttcttgacgagttcctgtctcgaaaaaag

SEQ ID NO: 14 - DNA, 21 bp, synthetic construct
  source 1..21: mol_type = other DNA; note = pLV-EF1a-MinE-F6E-EGFP-IRES-Blast; Baseline Circuit primer to SEQ ID 15 at complement(7698..7718)/label="pLV-EF1a-R"
tagagggaaaccgttgctagc

SEQ ID NO: 15 - DNA, 9545 bp, unidentified
  source 1..9545: mol_type = other DNA; note = pLV-EF1a-MinE-F6E-EGFP-IRES-Blast; Baseline Circuit
  CDS 6630..7646: protein_id = 16; translation = MALLDEFLSRKKNTANIAKERLQIIVAERRRSDAEPHYLPQLRK DILEVICKYVQIDPEMVTVQLEQKDGDISILELNVTLPEAEELKDSAGSAGSAGTMVS KGEELFTGVVPILVELDGDVNGHKFSVSGEGEGDATYGKLTLKFICTTGKLPVPWPTL VTTLTYGVQCFSRYPDHMKQHDFFKSAMPEGYVQERTIFFKDDGNYKTRAEVKFEGDT LVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYK
tggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggcccttcttgacgagttcctgtctcgaaaaaagaacacagcgaatatcgcgaaagaacggttgcaaattatagtggcggagagacgacgaagcgacgcggagcctcattaccttcctcaattgcgaaaagacatactggaagtgatctgcaagtacgttcaaattgaccctgagatggtgaccgtgcaacttgaacagaaggatggagatataagcatcctggaattgaacgtaacccttcccgaggcggaagaacttaaagacagcgccggaagtgctggctctgcgggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtcatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggac

SEQ ID NO: 16 - protein, 338 aa, unidentified
  source 1..338: note = pLV-EF1a-MinE-F6E-EGFP-IRES-Blast; Baseline Circuit CDS
MALLDEFLSRKKNTANIAKERLQIIVAERRRSDAEPHYLPQLRKDILEVICKYVQIDPEMVTVQLEQKDGDISILELNVTLPEAEELKDSAGSAGSAGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 21 bp, synthetic construct
  source 1..21: mol_type = other DNA; note = pLV-EF1a-MinE-GFP-P2A-mCherry-MinD-IRES-Puro; Baseline Circuit; primer to SEQ ID 23 at 3328..3348; EF1a-F
tcaagcctcagacagtggttc

SEQ ID NO: 18 - DNA, 49 bp, synthetic construct; identical to SEQ ID NO: 8
  source 1..49: mol_type = other DNA; note = pLV-EF1a-MinE-GFP-P2A-mCherry-MinD-IRES-Puro; Baseline Circuit; primer to SEQ ID 23 at 3356..3404; MinE-clip F

SEQ ID NO: 19 - DNA, 81 bp, synthetic construct
  source 1..81: mol_type = other DNA; note = pLV-EF1a-MinE-GFP-P2A-mCherry-MinD-IRES-Puro; Baseline Circuit; primer to SEQ ID 23 at complement(4389..4469); MinE-GFP-P2A R
gggaccggggttttcttccacgtcgcctgcttgctttaacagagagaagttcgtggcgctagccttgtacagctcgtccat

SEQ ID NO: 20 - DNA, 81 bp, synthetic construct; identical to SEQ ID NO: 19
  source 1..81: mol_type = other DNA; note = pLV-EF1a-MinE-GFP-P2A-mCherry-MinD-IRES-Puro; Baseline Circuit; primer to SEQ ID 23 at 4438..4487; P2A-mCherry-MinD F

SEQ ID NO: 21 - DNA, 49 bp, synthetic construct; identical to SEQ ID NO: 3
  source 1..49: mol_type = other DNA; note = pLV-EF1a-MinE-GFP-P2A-mCherry-MinD-IRES-Puro; Baseline Circuit; primer to SEQ ID 23 at complement(6006..6054); snap-MinD R

SEQ ID NO: 22 - DNA, 21 bp, synthetic construct
  source 1..21: mol_type = other DNA; note = pLV-EF1a-MinE-GFP-P2A-mCherry-MinD-IRES-Puro; Baseline Circuit; primer to SEQ ID 23 at complement(6075..6095); pLV-EF1a-R
tagagggaaaccgttgctagc

SEQ ID NO: 23 - DNA, 11330 bp, unidentified
  source 1..11330: mol_type = other DNA
  CDS 3393..6023: protein_id = 24; translation = MALLDFFLSRKKNTANIAKERLQIIVAERRRSDAEPHYLPQLRK DILEVICKYVQIDPEMVTVQLEQKDGDISILELNVTLPEAEELKDSAGSAGSAGTMVS KGEELFTGVVPILVELDGDVNGHKFSVSGEGEGDATYGKLTLKFICTTGKLPVPWPTL VTTLTYGVQCFSRYPDHMKQHDFFKSAMPEGYVQERTIFFKDDGNYKTRAEVKFEGDT LVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYKASATNFSLLKQAGDVEENPGPMVSKGEEDNMAIIKEFMRFKVHMEGSVNGHEFE IEGEGEGRPYEGTQTAKLKVTKGGPLPFAWDILSPQFMYGSKAYVKHPADIPDYLKLS FPEGFKWERVMNFEDGGVVTVTQDSSLQDGEFIYKVKLRGTNFPSDGPVMQKKTMGWE ASSERMYPEDGALKGEIKQRLKLKDGGHYDAEVKTTYKAKKPVQLPGAYNVNIKLDIT SHNEDYTIVEQYERAEGRHSTGGMDELYKDSAGSAGSAGTMARIIVVTSGKGGVGKTT SSAAIATGLAQKGKKTVVIDFDIGLRNLDLIMGCERRVVYDFVNVIQGDATLNQALIK DKRTENLYILPASQTRDKDALTREGVAKVLDDLKAMDFEFIVCDSPAGIETGALMALY FADEAIITTNPEVSSVRDSDRILGILASKSRRAENGEEPIKEHLLLTRYNPGRVSRGD MLSMEDVLEILRIKLVGVIPEDQSVLRASNQGEPVILDINADAGKAYADTVERLLGEE RPFRFIEEEKKGFLKRLFGG
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggcccttcttgacttcttcctgtctcgaaaaaagaacacagcgaatatcgcgaaagaacggttgcaaattatagtggcggagagacgacgaagcgacgcggagcctcattaccttcctcaattgcgaaaagacatactggaagtgatctgcaagtacgttcaaattgaccctgagatggtgaccgtgcaacttgaacagaaggatggagatataagcatcctggaattgaacgtaacccttcccgaggcggaagaacttaaagacagcgccggaagtgctggctctgcgggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggctagcgccacgaacttctctctgttaaagcaagcaggcgacgtggaagaaaaccccggtcccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggacagcgccggaagtgctggctctgcgggtaccatggcccgcataatagttgtgacttcaggtaagggcggagttggaaaaaccacatccagcgcagccatagctaccggcctcgcacagaaaggcaagaaaactgtggtcatagattttgatattgggctcaggaacctggacctgataatgggctgcgaacggcgcgttgtctacgattttgtcaatgtcatccaaggcgacgcgactcttaatcaggcacttataaaagataaacgaaccgaaaacttgtatattcttcctgcctcacaaacgcgggacaaagacgcgctcaccagggagggtgtcgcaaaagttttggatgacttgaaagcgatggactttgagttcatagtttgtgactctcccgccggtatagagacaggggcacttatggcactgtacttcgcagatgaagccataatcacaactaatccggaggtgtctagtgtgcgagattcagaccgaatactggggatactcgccagcaaatcacggagggcagaaaacggggaggagccaattaaggagcatctcctgcttacccgatataaccccggtcgagtatctcggggagatatgctttcaatggaagatgtcctcgagattctgaggataaaactggtaggcgttatcccggaagaccagtccgtcctccgcgcatctaatcagggggagcctgtcatacttgacataaatgcggatgctgggaaagcttatgccgatacggtagaacggctcctcggggaagaacggcccttccgcttcatagaggaggaaaagaagggattccttaaacggctgttcgggggataagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 24 - protein, 876 aa, unidentified
  source 1..876: note = pLV-EF1a-MinE-GFP-P2A-mCherry-MinD-IRES-Puro; Baseline Circuit; CDS
MALLDFFLSRKKNTANIAKERLQIIVAERRRSDAEPHYLPQLRKDILEVICKYVQIDPEMVTVQLEQKDGDISILELNVTLPEAEELKDSAGSAGSAGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDSAGSAGSAGTMARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 25 - DNA, 21 bp, synthetic construct
  source 1..21: mol_type = other DNA; note = pLV-EF1a-MinE-L4E-EGFP-IRES-Blast; Baseline Circuit; primer at 6565..6585; EF1a-F
tcaagcctcagacagtggttc

SEQ ID NO: 26 - DNA, 67 bp, synthetic construct
  source 1..67: mol_type = other DNA; note = pLV-EF1a-MinE-L4E-EGFP-IRES-Blast; Baseline Circuit; primer at 6593..6659; MinE_L4E
ttttcttccatttcaggtgtcgtgaggatccgccaccatggcccttgaggacttcttcctgtctcga

SEQ ID NO: 27 - DNA, 21 bp, synthetic construct
  source 1..21: mol_type = other DNA; note = pLV-EF1a-MinE-L4E-EGFP-IRES-Blast; Baseline Circuit; primer at complement(7698..7718); pLV-EF1a-R
tagagggaaaccgttgctagc

SEQ ID NO: 28 - DNA, 9545 bp, unidentified
  source 1..9545: mol_type = other DNA; note = pLV-EF1a-MinE-L4E-EGFP-IRES-Blast; Baseline Circuit
  CDS 6630..7646: note = pLV-EF1a-MinE-L4E-EGFP-IRES-Blast; Baseline Circuit CDS; protein_id = 29; translation = MALEDFFLSRKKNTANIAKERLQIIVAERRRSDAEPHYLPQLRK DILEVICKYVQIDPEMVTVQLEQKDGDISILELNVTLPEAEELKDSAGSAGSAGTMVS KGEELFTGVVPILVELDGDVNGHKFSVSGEGEGDATYGKLTLKFICTTGKLPVPWPTL VTTLTYGVQCFSRYPDHMKQHDFFKSAMPEGYVQERTIFFKDDGNYKTRAEVKFEGDT LVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYK
tggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggcccttgaggacttcttcctgtctcgaaaaaagaacacagcgaatatcgcgaaagaacggttgcaaattatagtggcggagagacgacgaagcgacgcggagcctcattaccttcctcaattgcgaaaagacatactggaagtgatctgcaagtacgttcaaattgaccctgagatggtgaccgtgcaacttgaacagaaggatggagatataagcatcctggaattgaacgtaacccttcccgaggcggaagaacttaaagacagcgccggaagtgctggctctgcgggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtcatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggac

SEQ ID NO: 29 - protein, 338 aa, unidentified
  source 1..338: note = pLV-EF1a-MinE-L4E-EGFP-IRES-Blast; Baseline Circuit CDS
MALEDFFLSRKKNTANIAKERLQIIVAERRRSDAEPHYLPQLRKDILEVICKYVQIDPEMVTVQLEQKDGDISILELNVTLPEAEELKDSAGSAGSAGTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - DNA, 21 bp, synthetic construct
  source 1..21: mol_type = other DNA; note = pLV-EF1a-PKAsub-mCherry-MinD-IRES-Puro:Broadcast Circuit -Activity Circuit; primer of SEQ ID 34 at 3328..3348; EF1a-F
tcaagcctcagacagtggttc

SEQ ID NO: 31 - DNA, 87 bp, synthetic construct
  source 1..87: mol_type = other DNA; note = pLV-EF1a-PKAsub-mCherry-MinD-IRES-Puro:Broadcast Circuit -Activity Circuit; primer of SEQ ID 34 at 3351..3437; PKAsub_mCherry_F
agtttttttcttccatttcaggtgtcgtgaggatccgccaccatgttgaggcgcgcgacgctggttgacatggtgagcaagggcgag

SEQ ID NO: 32 - DNA, 49 bp, synthetic construct; identical to SEQ ID NO: 3
  source 1..49: mol_type = other DNA; note = pLV-EF1a-PKAsub-mCherry-MinD-IRES-Puro:Broadcast Circuit -Activity Circuit; primer of SEQ ID 34 at complement(4956..5004); snap-MinD R

SEQ ID NO: 33 - DNA, 21 bp, synthetic construct
  source 1..21: mol_type = other DNA; note = pLV-EF1a-PKAsub-mCherry-MinD-IRES-Puro:Broadcast Circuit -Activity Circuit; primer of SEQ ID 34 at complement(5025..5045); pLV-EF1a-R
tagagggaaaccgttgctagc

SEQ ID NO: 34 - DNA, 10280 bp, unidentified
  source 1..10280: mol_type = other DNA; note = pLV-EF1a-PKAsub-mCherry-MinD-IRES-Puro:Broadcast Circuit -Activity Circuit
  CDS 3393..4973: protein_id = 35; translation = MLRRATLVDMVSKGEEDNMAIIKEFMRFKVHMEGSVNGHEFEIE GEGEGRPYEGTQTAKLKVTKGGPLPFAWDILSPQFMYGSKAYVKHPADIPDYLKLSFP EGFKWERVMNFEDGGVVTVTQDSSLQDGEFIYKVKLRGTNFPSDGPVMQKKTMGWEAS SERMYPEDGALKGEIKQRLKLKDGGHYDAEVKTTYKAKKPVQLPGAYNVNIKLDITSH NEDYTIVEQYERAEGRHSTGGMDELYKDSAGSAGSAGTMARIIVVTSGKGGVGKTTSS AAIATGLAQKGKKTVVIDFDIGLRNLDLIMGCERRVVYDFVNVIQGDATLNQALIKDK RTENLYILPASQTRDKDALTREGVAKVLDDLKAMDFEFIVCDSPAGIETGALMALYFA DEAIITTNPEVSSVRDSDRILGILASKSRRAENGEEPIKEHLLLTRYNPGRVSRGDML SMEDVLEILRIKLVGVIPEDQSVLRASNQGEPVILDINADAGKAYADTVERLLGEERP FRFIEEEKKGFLKRLFGG
  CDS 4161..4970: protein_id = 36; translation = MARIIVVTSGKGGVGKTTSSAAIATGLAQKGKKTVVIDFDIGLR NLDLIMGCERRVVYDFVNVIQGDATLNQALIKDKRTENLYILPASQTRDKDALTREGV AKVLDDLKAMDFEFIVCDSPAGIETGALMALYFADEAIITTNPEVSSVRDSDRILGIL ASKSRRAENGEEPIKEHLLLTRYNPGRVSRGDMLSMEDVLEILRIKLVGVIPEDQSVL RASNQGEPVILDINADAGKAYADTVERLLGEERPFRFIEEEKKGFLKRLFGG
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatgttgaggcgcgcgacgctggttgacatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggacagcgccggaagtgctggctctgcgggtaccatggcccgcataatagttgtgacttcaggtaagggcggagttggaaaaaccacatccagcgcagccatagctaccggcctcgcacagaaaggcaagaaaactgtggtcatagattttgatattgggctcaggaacctggacctgataatgggctgcgaacggcgcgttgtctacgattttgtcaatgtcatccaaggcgacgcgactcttaatcaggcacttataaaagataaacgaaccgaaaacttgtatattcttcctgcctcacaaacgcgggacaaagacgcgctcaccagggagggtgtcgcaaaagttttggatgacttgaaagcgatggactttgagttcatagtttgtgactctcccgccggtatagagacaggggcacttatggcactgtacttcgcagatgaagccataatcacaactaatccggaggtgtctagtgtgcgagattcagaccgaatactggggatactcgccagcaaatcacggagggcagaaaacggggaggagccaattaaggagcatctcctgcttacccgatataaccccggtcgagtatctcggggagatatgctttcaatggaagatgtcctcgagattctgaggataaaactggtaggcgttatcccggaagaccagtccgtcctccgcgcatctaatcagggggagcctgtcatacttgacataaatgcggatgctgggaaagcttatgccgatacggtagaacggctcctcggggaagaacggcccttccgcttcatagaggaggaaaagaagggattccttaaacggctgttcgggggataagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 35 - protein, 526 aa, unidentified
  source 1..526: note = pLV-EF1a-PKAsub-mCherry-MinD-IRES-Puro:Broadcast Circuit -Activity Circuit CDS
MLRRATLVDMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDSAGSAGSAGTMARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 36 - protein, 270 aa, unidentified; identical to SEQ ID NO: 6
  source 1..270: note = pLV-EF1a-PKAsub-mCherry-MinD-IRES-Puro:Broadcast Circuit -Activity Circuit CDS

SEQ ID NO: 37 - DNA, 8527 bp, unidentified
  source 1..8527: mol_type = other DNA; note = ActA(1-584)-BFP-FKBP in pTwist Lenti SFFV; Payload Circuit
  CDS 7..2844: protein_id = 38; translation = MGLNRFMRAMMVVFITANCITINPDIIFAATDSEDSSLNTDEWE EEKTEEQPSEVNTGPRYETAREVSSRDIKELEKSNKVRNTNKADLIAMLKEKAEKGPN INNNNSEQTENAAINEEASGADRPAIQVERRHPGLPSDSAAEIKKRRKAIASSDSELE SLTYPDKPTKVNKKKVAKESVADASESDLDSSMQSADESSPQPLKANQQPFFPKVFKK IKDAGKWVRDKIDENPEVKKAIVDKSAGLIDQLLTKKKSEEVNASDFPPPPTDEELRL ALPETPMLLGFNAPATSEPSSFEFPPPPTDEELRLALPETPMLLGFNAPATSEPSSFE FPPPPTEDELEIIRETASSLDSSFTRGDLASLRNAINRHSQNFSDFPPIPTEEELNGR GGRPTSEEFSSLNSGDFTDDENSETTEEEIDRLADLRDRGTGKHSRNAGFLPLNPFAS SPVPSLSPKVSKISAPALISDITKKTPFKNPSQPLNVFNKKTTTKTVTKKPTPVKTAP KLAELPATKPQETVLRENKTPFIEKQAETNKQSINMPSLPVIQKEATESDKEEMKPQT EEKMVEESESANNANGKNGSGGSGGGGSMSELIKENMHMKLYMEGTVDNHHFKCTSEG EGKPYEGTQTMRIKVVEGGPLPFAFDILATSFLYGSKTFINHTQGIPDFFKQSFPEGF TWERVTTYEDGGVLTATQDTSLQDGCLIYNVKIRGVNFTSNGPVMQKKTLGWEAFTET LYPADGGLEGRNDMALKLVGGSHLIANIKTTYRSKKPAKNLKMPGVYYVDYRLERIKE ANNETYVEQHEVAVARYCDLPSKLGHKLNDSAGSAGSAGTGVQVETISPGDGRTFPKR GQTCVVHYTGMLEDGKKFDSSRDRNKPFKFMLGKQEVIRGWEEGVAQMSVGQRAKLTI SPDYAYGATGHPGIIPPHATLVFDVELLKLE
  CDS 2521..2844: protein_id = 39; translation = GVQVETISPGDGRTFPKRGQTCVVHYTGMLEDGKKFDSSRDRNK PFKFMLGKQEVIRGWEEGVAQMSVGQRAKLTISPDYAYGATGHPGIIPPHATLVFDVE LLKLE
gccaccatgggactgaacagatttatgcgggccatgatggtggttttcattactgccaactgcattacgattaaccccgacataatattcgcagcgactgacagcgaagattctagtttgaacacagacgaatgggaggaggaaaagacagaagagcagcccagcgaggtgaataccggaccacgctacgaaactgcacgtgaggtgagttcacgggatattaaagaactcgagaaaagcaataaggtgagaaacactaacaaagcagacctaatcgcaatgttgaaagaaaaagccgagaaaggtccaaacatcaataataacaacagtgaacagactgagaatgccgctataaatgaagaggcttcaggagccgaccgacccgctatccaggtggagcgtcggcaccctggcctgccctccgacagcgccgccgaaattaaaaagaggaggaaagccatcgcatcctcggatagtgagcttgaaagcctgacttatccggataaaccaacaaaagtaaataagaagaaggtggcgaaggagtcagttgccgatgcttctgaaagtgacttagactctagcatgcagtcagcagacgagtcttccccacaacctctgaaagccaaccaacagcccttcttccctaaggtctttaaaaaaataaaggatgccgggaaatgggtccgcgacaagatcgacgaaaatcctgaggtcaagaaggcgattgttgataaatccgccgggctcatcgaccagctgctcaccaagaagaagagtgaggaggtgaatgcttctgacttcccgcccccacctacggatgaagagctgcgactggctttgccagagacccctatgcttctggggtttaatgctcctgccacatcagaaccgagctcattcgagtttccaccaccccctaccgacgaagagctgaggcttgctttgcccgagacgccaatgctgcttggttttaatgcccctgccacctccgagcccagctcgttcgaattcccacctcctcccactgaggatgaactcgagatcatccgggagacagcatcctctctcgattcttcctttacccgcggggacttagcttccttgcgaaatgctattaaccgccattcccaaaatttctctgacttcccacctatccccacagaagaggagttgaacgggaggggcggcagaccaacatctgaggagttctcctcgctgaatagtggtgattttaccgatgacgagaacagcgagacaacagaagaagaaattgatcgcctagccgatctgagagatagaggaacaggaaaacactcaaggaatgcgggatttctgcccctgaatccgttcgccagcagcccggttccttctttaagtcccaaggtctccaaaatcagcgcaccggctctgatcagtgacataactaaaaagacgccatttaagaatccttcccagcccctgaatgtgtttaataagaagactaccactaagaccgtgactaagaaacctacccctgtgaagaccgccccaaagctagcagaacttcctgccacaaagccccaggagaccgtacttagggaaaacaagaccccctttatagagaagcaggcagagacaaacaagcagtcaatcaacatgcccagcctccccgtcatccagaaagaggctaccgagagcgataaggaggagatgaaaccacaaaccgaggaaaagatggtagaggaaagcgaatcagccaacaacgcaaacggcaagaacggtagcggcggaagcggtggcggtggcagcatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgacagcgccggaagtgctggctctgcgggtaccggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatgactcgagggcccgcatccaccatcgcagacttatcatcttattccagatgcgtgcggatggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaacgcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcattgtcgatcctaccatccactcgacacacccgccaggatcc

SEQ ID NO: 38 - protein, 945 aa, unidentified
  source 1..945: note = ActA(1-584)-BFP-FKBP in pTwist Lenti SFFV; Payload Circuit CDS
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISAPALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNGSGGSGGGGSMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNDSAGSAGSAGTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 39 - protein, 107 aa, unidentified
  source 1..107: note = ActA(1-584)-BFP-FKBP in pTwist Lenti SFFV; Payload Circuit CDS
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 40 - DNA, 10181 bp, unidentified
  source 1..10181: mol_type = other DNA; note = pLV-EF1a-BFP-FKBP-IRES-Hygro; Payload Circuit
  CDS 3393..4448: protein_id = 41; translation = MSELIKENMHMKLYMEGTVDNHHFKCTSEGEGKPYEGTQTMRIK VVEGGPLPFAFDILATSFLYGSKTFINHTQGIPDFFKQSFPEGFTWERVTTYEDGGVL TATQDTSLQDGCLIYNVKIRGVNFTSNGPVMQKKTLGWEAFTETLYPADGGLEGRNDM ALKLVGGSHLIANIKTTYRSKKPAKNLKMPGVYYVDYRLERIKEANNETYVEQHEVAV ARYCDLPSKLGHKLNDSAGSAGSAGTGVQVETISPGDGRTFPKRGQTCVVHYTGMLED GKKFDSSRDRNKPFKFMLGKQEVIRGWEEGVAQMSVGQRAKLTISPDYAYGATGHPGI IPPHATLVFDVELLKLE
  CDS 4125..4448: protein_id = 42; translation = GVQVETISPGDGRTFPKRGQTCVVHYTGMLEDGKKFDSSRDRNK PFKFMLGKQEVIRGWEEGVAQMSVGQRAKLTISPDYAYGATGHPGIIPPHATLVFDVE LLKLE
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgacagcgccggaagtgctggctctgcgggtaccggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatgagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 41 - protein, 351 aa, unidentified
  source 1..351: note = pLV-EF1a-BFP-FKBP-IRES-Hygro; Payload Circuit CDS
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNDSAGSAGSAGTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 42 - protein, 107 aa, unidentified; identical to SEQ ID NO: 39
  source 1..107: note = pLV-EF1a-BFP-FKBP-IRES-Hygro; Payload Circuit CDS

SEQ ID NO: 43 - DNA, 21 bp, synthetic construct
  source 1..21: mol_type = other DNA; note = pLV-EF1a-FRB-mCherry-MinD-IRES-Puro; Payload Circuit; primer at 3328..3348; EF1a-F
tcaagcctcagacagtggttc

SEQ ID NO: 44 - DNA, 58 bp, synthetic construct
  source 1..58: mol_type = other DNA; note = pLV-EF1a-FRB-mCherry-MinD-IRES-Puro; Payload Circuit; primer at 3356..3413; FRB_mCherryMinD_F
ttttcttccatttcaggtgtcgtgaggatccgccaccatggagatgtggcatgaaggc

SEQ ID NO: 45 - DNA, 43 bp, synthetic construct
  source 1..43: mol_type = other DNA; note = pLV-EF1a-FRB-mCherry-MinD-IRES-Puro; Payload Circuit; primer at complement(3648..3690); FRB_mCherryMinD_R
tatcctcctcgcccttgctcaccatctgctttgagattcgtcg

SEQ ID NO: 46 - DNA, 18 bp, synthetic construct
  source 1..18: mol_type = other DNA; note = pLV-EF1a-FRB-mCherry-MinD-IRES-Puro; Payload Circuit; primer at 3666..3683; mCherry F
atggtgagcaagggcgag

SEQ ID NO: 47 - DNA, 49 bp, synthetic construct; identical to SEQ ID NO: 3
  source 1..49: mol_type = other DNA; note = pLV-EF1a-FRB-mCherry-MinD-IRES-Puro; Payload Circuit; primer at complement(5202..5250); snap-MinD R

SEQ ID NO: 48 - DNA, 21 bp, synthetic construct
  source 1..21: mol_type = other DNA; note = pLV-EF1a-FRB-mCherry-MinD-IRES-Puro; Payload Circuit; primer at complement(5271..5291); pLV-EF1a-R
tagagggaaaccgttgctagc

SEQ ID NO: 49 - DNA, 10526 bp, unidentified
  source 1..10526: mol_type = other DNA; note = pLV-EF1a-FRB-mCherry-MinD-IRES-Puro; Payload Circuit
  CDS 3396..3665: protein_id = 50
  CDS 3666..4373: protein_id = 51; translation = MVSKGEEDNMAIIKEFMRFKVHMEGSVNGHEFEIEGEGEGRPYE GTQTAKLKVTKGGPLPFAWDILSPQFMYGSKAYVKHPADIPDYLKLSFPEGFKWERVM NFEDGGVVTVTQDSSLQDGEFIYKVKLRGTNFPSDGPVMQKKTMGWEASSERMYPEDG ALKGEIKQRLKLKDGGHYDAEVKTTYKAKKPVQLPGAYNVNIKLDITSHNEDYTIVEQ YERAEGRHSTGGMDELYK
  CDS 4374..4406: protein_id = 52; translation = DSAGSAGSAGT
  CDS 4407..5216: protein_id = 53; translation = MARIIVVTSGKGGVGKTTSSAAIATGLAQKGKKTVVIDFDIGLR NLDLIMGCERRVVYDFVNVIQGDATLNQALIKDKRTENLYILPASQTRDKDALTREGV AKVLDDLKAMDFEFIVCDSPAGIETGALMALYFADEAIITTNPEVSSVRDSDRILGIL ASKSRRAENGEEPIKEHLLLTRYNPGRVSRGDMLSMEDVLEILRIKLVGVIPEDQSVL RASNQGEPVILDINADAGKAYADTVERLLGEERPFRFIEEEKKGFLKRLFGG
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgaggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccaccatggagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggacagcgccggaagtgctggctctgcgggtaccatggcccgcataatagttgtgacttcaggtaagggcggagttggaaaaaccacatccagcgcagccatagctaccggcctcgcacagaaaggcaagaaaactgtggtcatagattttgatattgggctcaggaacctggacctgataatgggctgcgaacggcgcgttgtctacgattttgtcaatgtcatccaaggcgacgcgactcttaatcaggcacttataaaagataaacgaaccgaaaacttgtatattcttcctgcctcacaaacgcgggacaaagacgcgctcaccagggagggtgtcgcaaaagttttggatgacttgaaagcgatggactttgagttcatagtttgtgactctcccgccggtatagagacaggggcacttatggcactgtacttcgcagatgaagccataatcacaactaatccggaggtgtctagtgtgcgagattcagaccgaatactggggatactcgccagcaaatcacggagggcagaaaacggggaggagccaattaaggagcatctcctgcttacccgatataaccccggtcgagtatctcggggagatatgctttcaatggaagatgtcctcgagattctgaggataaaactggtaggcgttatcccggaagaccagtccgtcctccgcgcatctaatcagggggagcctgtcatacttgacataaatgcggatgctgggaaagcttatgccgatacggtagaacggctcctcggggaagaacggcccttccgcttcatagaggaggaaaagaagggattccttaaacggctgttcgggggataagcggccgctctagagtcgacgggccgcggtaacaattgttaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacccgggcggggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 50 - protein, 90 aa, unidentified [sequence omitted]

SEQ ID NO: 51 - protein, 236 aa, unidentified
  source 1..236: note = pLV-EF1a-FRB-mCherry-MinD-IRES-Puro; Payload Circuit CDS
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 52 - protein, 11 aa, unidentified
  source 1..11: note = pLV-EF1a-FRB-mCherry-MinD-IRES-Puro; Payload Circuit CDS
DSAGSAGSAGT

SEQ ID NO: 53 - protein, 270 aa, unidentified; identical to SEQ ID NO: 6
  source 1..270: note = pLV-EF1a-FRB-mCherry-MinD-IRES-Puro; Payload Circuit CDS

SEQ ID NO: 54 - protein, 270 aa, Escherichia coli; identical to SEQ ID NO: 6
  source 1..270: note = Escherichia_coli- MinD

SEQ ID NO: 55 - protein, 270 aa, Fukatsuia symbiotica
  source 1..270: note = Fukatsuia_symbiotica- MinD
MARIIVVTSGKGGVGKTTSSAAIATGLAQQGKKTVVIDFDIGLRNLDLIMGCERRVVYDFVNVIQEDATLNQALIKDKRTENLYILPASQTRDKDALTKEGVEKVLTGLDEMNFEYVVCDSPAGIETGALMALYFADEAIITTNPEVSSVRDSDRILGILSSKSRRAKRGEEPIKEHLLLTRYNPSRVSRGDMLSMEDVLDILRIPLLGVIPEDQSVLSASNQGEPVILNGGSNAGKAYADTVARLLGEKRNFRFIEEEKKGFLKRLFGG

SEQ ID NO: 56 - protein, 270 aa, Gullanella endobia
  source 1..270: note = Gullanella_endobia- MinD
MVRIIVVTSGKGGVGKTTSSAAIATGLARKGKKTVVIDFDIGLRNLDLIMGCERRVVYDFINVINCDATLNQALIKDKRTENLYVLPASQTRDKDALTREGVEKVLNDLDTMEFDFIICDSPAGIETGALMALYFADEAIITTNPEISSVRDSDRILGILSAKSRRAENGLEAIKEHLLLTRYNPRRVKCGDMLSMRDVIEILRIPLLGLIPEDQSVLRASNQGEPVILDKESDAGQAYIDMVDRLLGEEHPFRFIEEEKKGFIKRLFRG

SEQ ID NO: 57 - protein, 271 aa, Moranella endobia
  source 1..271: note = Moranella_endobia- MinD
MARIIVVTSGKGGVGKTTSSASIATGLARKGKKTVVIDFDIGLRNLDLIMGCERRVVYDFVNVVQGAARLTQALIKDKRTDNLYILPASQTKDQDALTCAGVEKLLNDLNKMEFDFIVCDSPAGIETGALMALYFADEAIITTNPEVSSVRDSDRILGILSSKSRRAEIGQEPIKEHLLLNRYNPGRVSRGDMLSIDDVIEILRIPLVGVIPEDQSVLQASNQGEPVILNEDSDAGQAYSDMVDRLLGQECPFRFIAEPQKKSLLKRLFGV

SEQ ID NO: 58 - protein, 264 aa, Clostridium tertium
  source 1..264: note = Clostridium_tertium- MinD
MGVSIVITSGKGGVGKTTTTANIGTALAALNKRVVVVDGDTGLRNLDVLMGLENRIVYTITDVIENRCRLKQALIKDKRYQNLCLLPTAQTKDKDDIRPQDMLKLINELKEDFDYVLIDCPAGIEQGFENSVVGADRAVVVVNPEITSVRDADRVIGKLDAKGLDDHAVIINRLNYEMTQRGDMLDVSDIIETLSIELLGVVPDDKNITVSTNKGEPIVLDDKSISGQAFKNIARRITGEEVPLLDLKTGGEGFFASIKRLFKR

SEQ ID NO: 59 - protein, 270 aa, Dickeya dadantii
  source 1..270: note = Dickeya_dadantii- MinD
MARIIVVTSGKGGVGKTTSSAAIATGLAQKGKKTVVIDFDIGLRNLDLIMGCERRVVYDFVNVIQNDATLNQALIKDKRTENLYILPASQTRDKEALTREGVDKVLKDLADMAFDFIICDSPAGIETGALMALYFADEAIITTHPEVSSVRDSDRILGILSSKSRRAEQGQEPIKEHLLLTRYNPGRVSRGDMLSMEDVLEILRIPLVGVIPEDQSVLRASNQGEPVILDKDADAGKAYEDTVDRLLGEERPYRFIEEEKKSFLKRLFGG

SEQ ID NO: 60 - protein, 263 aa, Helicobacter felis
  source 1..263: note = Helicobacter_felis- MinD
MVITITSGKGGVGKSTTTANLAIGLALQNKKVVAVDFDIGLRNLDMILGLENRIVYDVIDVMEGNCKLPQALINDKKNKNLYFLPASQSKDKNILDKAKVQALIAQLNAQFDFVLIDSPAGIESGFEHAVLFADRAIIVVTPEVSSVRDSDRVIGIIDAKSCKGQEMVKHILINRIKPDLVEKQEMLSNEDVLKILALPLIGLVPEDDKIVSATNTGEPVIYTQSPSALAFQRITRRVLGEEVEFAEFRTKRGLVGTIKGWFA

SEQ ID NO: 61 - protein, 268 aa, Helicobacter pylori
  source 1..268: note = Helicobacter_pylori- MinD
MAIVVTITSGKGGVGKSTTTANLAIGLAESGKKVVAVDFDIGLRNLDMILGLENRIVYDVVDVMEKNCNLSQALITDKKTKNLSFLAASQSKDKNILDKEKVAILINALRADFDYILIDSPAGIESGFEHAILHADMALVVVTPEVSSLRDSDRVIGIIDAKSNRAKKGMEVHKHLIINRLKPELVANGEMISIEEVLKILCLPLIGIIPEDSHIISATNKGEPVIRADCESAKAYQRITRRILGEEVEYVEFKAKRGFFGALKGIFS

SEQ ID NO: 62 - protein, 268 aa, Klebsiella quasipneumoniae
  source 1..268: note = Klebsiella_quasipneumoniae- MinD
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVDKVLEELKKMEFDFIVCDSPAGIETGALMALYFADEAIITTNPEVSSVRDSDRILGILASKSRRAENGEEPIKEHLLLTRYNPGRVNKGDMLSMEDVLEILRINLVGVIPEDQSVLRASNQGEPVILDAASDAGKAYADTVERLLGEERPFRFIEEEKKGFLKRLF

SEQ ID NO: 63 - protein, 270 aa, Pantoea sp.Mhis
  source 1..270: note = Pantoea_sp.Mhis- MinD
MSRIIVVTSGKGGVGKTTSSAAIATGLAQNNKRTAVIDFDIGLRNLDLVMGCERRVVYDFINVIQGDATLNQALIRDKHTEQLYILPASQTRNKDALTRKGVEKVIQELEENDFDFIICDSPAGIEAGALMALYFADEAIIITNPEVSSVHDSDRILGIISSKSYRAENGKTPIKEYLLLTRYNPNRVTRGDMLSMEDVLGILRIPLLGVIPEDQSVLRSSNQGEPVILDTNSDAGKAYFDTVERLLGKELPFRFIEEEKKGFIKRLFGG

SEQ ID NO: 64 - protein, 270 aa, Photobacterium damselae
  source 1..270: note = Photobacterium_damselae- MinD
MARIIVVTSGKGGVGKTTSSAAIASGLALRGKKTAVIDFDIGLRNLDLIMGCERRVVYDFVNVINGEANLNQALIKDKRTDNLFVLPASQTRDKDALSKEGVERVLKDLGEMGFDFVICDSPAGIETGALMALYFADEAIVTTNPEVSSVRDSDRILGILDSKSRRAEQGQEPVKQHLLLTRYNPTRVNQGDMLSVQDVEEILHIPLLGVIPESQAVLNASNKGEPVIFDKDADASIAYQDTVARLLGEECPFRFLEEEKKGFLKRLFGG

SEQ ID NO: 65 - protein, 270 aa, Pseudomonas poae
  source 1..270: note = Pseudomonas_poae- MinD
MAKILVVTSGKGGVGKTTTSAAIGTGLALRGHKTVIVDFDVGLRNLDLIMGCERRVVYDFVNVVNGEANLQQALIKDKRLENLYVLAASQTRDKDALTKEGVGKVLAELKETFEYVVCDSPAGIETGAHLAMYFADEAIVVTNPEVSSVRDSDRMLGLLASKSQRAEKGEEPIKEHLLLTRYNPERVNNGEMLGVEDVKDILAVTLLGVIPESQAVLKASNQGVPVILDDQSDAGQAYSDAVDRLLGKTVDHRFLDVKKKGFFERIFGGN

SEQ ID NO: 66 - protein, 270 aa, Thorsellia anophelis
  source 1..270: note = Thorsellia_anophelis- MinD
MTKVLVVTSGKGGVGKTTSSAAISTGLARKGKKTVVIDFDIGLRNLDLIMGCERRVVYDFVNVIQGDATLNQALIKDKRTENLYILPASQTRDKDALTRDGVEKVINELKEMEFDFIICDSPAGIESGALMALYFADEAIITTNPEVSSVRDSDRILGILSSKSKRAEQGDNPIKEHLLITRYNPTRVSHGDMLSMEDVLEILRVPLVGLIPEDQSVLRASNQGEPVILDENSDAGKAYDDMVARLLGEERQFRFLSEEKKSFLKRLFGG

SEQ ID NO: 67 - protein, 88 aa, Escherichia coli
  source 1..88: note = Escherichia_coli- Min E
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 68 - protein, 89 aa, Fukatsuia symbiotica
  source 1..89: note = Fukatsuia_symbiotica- Min E
MALLDFFLSGKKPTANIAKERLQIIVAERRRGEQEPHYLPDLKRDVLAVICKYVQINPDMLQVQFEQKGDDISVLELNVTLPDMEETSK

SEQ ID NO: 69 - protein, 88 aa, Gullanella endobia
  source 1..88: note = Gullanella_endobia- Min E
MALLNFFISRKKSTANIAKERLQIIVAEQRRGNNEPHYLPQLKRDLIEVINKYVQIDSKMLSMQLEKDGNISILELNIALLETEESIE

SEQ ID NO: 70 - protein, 84 aa, Moranella endobia
  source 1..84: note = Moranella_endobia- Min E
MVLIDFFLARKKNTANIAKERLHSIVAERRGSNEPYYLPQLKSDLLEVISKYAKIDITMISIQLDQKDENLSILELNVKLTDTH

SEQ ID NO: 71 - protein, 86 aa, Clostridium tertium
  source 1..86: note = Clostridium_tertium- Min E
MEFLRKLSSRPTPKEVAKDRLKLILIHDRGDLPHETLEKIRMEILEVLSKYIEIDSEDVEIAVSKTENVEGNNPALVANIPIKNIK

SEQ ID NO: 72 - protein, 93 aa, Dickeya dadantii
  source 1..93: note = Dickeya_dadantii- Min E
MALLDFFLSRKKTTANIAKERLQIIVAERRRGDSEPHYLPQLKRDILEVICKYVQIDPEMVTVQLEQKGDDISVLELNVTLPEADEATPPTDK

SEQ ID NO: 73 - protein, 80 aa, Helicobacter felis
  source 1..80: note = Helicobacter_felis- Min E
MKWFKGGSSARARDRLTLVLAYERSMRIPYMEEMKKEILAVVQKYIATTKIDVRTSSNQEMDTLEVEIILERNSKGNPES

SEQ ID NO: 74 - protein, 77 aa, Helicobacter pylori
  source 1..77: note = Helicobacter_pylori- Min E
MSLFDFFKSKGSAATATDRLKLILAKERTLNLPYMEEMRKEIIAVIQKYTKSSDIHFKTIDGNQSVETIEVEIILPK

SEQ ID NO: 75 - protein, 89 aa, Klebsiella quasipneumoniae
  source 1..89: note = Klebsiella_quasipneumoniae- Min E
MALLDFFLSRKKNTANIAKERLQIIVAERRRGDAEPHYLPQLRKDILEVICKYVQIDPEMVSVQLEQRDGDISILELNVTLPETEESKP

SEQ ID NO: 76 - protein, 93 aa, Pantoea sp.Mhis
  source 1..93: note = Pantoea_sp.Mhis- Min E
MALLKFFLSHKKNTANIAKERLQIIVAEHRKARNEPHYLPQLKRDILKVICKYVKINPEMVTVQLEHKQDNISILELNIALLEVKDLSNSNMI

SEQ ID NO: 77 - protein, 87 aa, Photobacterium damselae
  source 1..87: note = Photobacterium_damselae- Min E
MALLEFFRPKKTTTASVAKERLQIIVAERRSAGQGAPSYLPQLKQDILEVIRKYVAIDPEQVVVTLDQKEEDLAVLELNVTLPEEDK

SEQ ID NO: 78 - protein, 84 aa, Pseudomonas poae
  source 1..84: note = Pseudomonas_poae- Min E
MKFLDFFRANKKPSTASVAKERLQIIVAHERGQRSTPDYLPALQKELVEVIRKYVNIGNDDVHVALENDGSCSILELNITLPDR

SEQ ID NO: 79 - protein, 87 aa, Thorsellia anophelis
  source 1..87: note = Thorsellia_anophelis- Min E
MSLINFFLSKKKNTANIAKERLQIIVAERRKADSAPAYLEEMKRDLLAVICKYVQIDPDMLHVGYEQKDDDISVLELNITLPENEEK